(19) United States
(12) Patent Application Publication
(10) Pub. No.: US 2022/0372098 A1
(43) Pub. Date: Nov. 24, 2022

(54) METHODS AND MATERIALS FOR TARGETED EXPANSION OF IMMUNE EFFECTOR CELLS

(71) Applicants: The Johns Hopkins University, Baltimore, MD (US); Institute of Microbiology of the CAS, v. v. i., Prague (CZ)

(72) Inventors: Jamie Spangler, Baltimore, MD (US); Jakub Tomala, Baltimore, MD (US); Michael Isaac Leff, Baltimore, MD (US); Seth Ludwig, Baltimore, MD (US); Elissa Kathleen Leonard, Baltimore, MD (US); Marek Kovár, Prague (CZ); Jirina Kovárová, Prague (CZ)

(21) Appl. No.: 17/622,539

(22) PCT Filed: Jun. 26, 2020
(86) PCT No.: PCT/US2020/039857

§ 371 Date: Dec. 23, 2021

Related U.S. Application Data
(60) Provisional application No. 62/867,010, filed on Jun. 26, 2019.

(51) Int. Cl. C07K 14/55; C07K 16/24; A61P 35/00

(52) U.S. Cl. CPC C07K 14/55; C07K 16/246; A61P 35/00; C07K 2319/30; C07K 2317/622; A61K 2039/505

(57) ABSTRACT

This document relates to methods and materials for targeted expansion of immune effector (Eff) T cells. For example, a composition containing one or more amino acid chains (e.g., one or more single-chain antibody/cytokine fusion proteins (immunocytokines)) that can bind to a heterodimeric receptor including an interleukin-2 receptor-β (IL-2Rβ) polypeptide and a common gamma chain (γ_{c}) polypeptide (e.g., an IL-2Rβ/γ_{c} polypeptide complex) can be administered to a mammal to stimulate Effs within the mammal to activate an immune response in that mammal. In some cases, methods and materials that can be used to treat a mammal having a condition that can benefit from activating an immune response (e.g., a cancer and/or an infectious disease) are provided. For example, a composition containing one or more single-chain immunocytokines that can bind to an IL-2Rβ/γ_{c} polypeptide complex can be administered to a mammal having a cancer and/or an infectious disease to treat the mammal.

### CROSS-REFERENCE TO RELATED APPLICATIONS

This application claims the benefit of U.S. Patent Application Ser. No. 62/867,010, filed on Jun. 26, 2019. The disclosure of the prior application is considered part of (and is incorporated by reference in) the disclosure of this application.

### BACKGROUND

### 1. Technical Field

This document relates to methods and materials for targeted expansion of immune effector cells (Effs). For example, a composition containing one or more amino acid chains (e.g., one or more single-chain antibody/cytokine fusion proteins (immunocytokines)) that can bind to a heterodimeric receptor including an interleukin-2 receptor-β (IL-2Rβ) polypeptide and a common gamma chain (γc) polypeptide (e.g., an IL-2Rβ/γ_{c} polypeptide complex) can be administered to a mammal to stimulate Effs cells within the mammal to activate an immune response in that mammal. In some cases, methods and materials provided herein can be used to treat a mammal having a condition that can benefit from activating an immune response (e.g., a cancer and/or an infectious disease). For example, a composition containing one or more single-chain immunocytokines that can bind to an IL-2Rβ/γ_{c} polypeptide complex can be administered to a mammal having a cancer and/or an infectious disease to treat the mammal.

### 2. Background Information

IL-2 is a multi-functional cytokine that orchestrates the differentiation, proliferation, survival, and activity of immune cells. Due to its potent activation of the immune response, high-dose IL-2 therapy has been used clinically to stimulate anti-cancer immunity and received FDA approval for treatment of metastatic renal cell carcinoma in 1992 and for metastatic melanoma in 1998 (Liao et al., Immunity. 38(1):13-25 (2013)). By activating the patient's own immune system, IL-2 therapy elicits complete and durable responses in 5-10% of patients (Rosenberg et al., Sci Transl Med. 4(127):127ps8 (2012)). However, IL-2 simultaneously activates both Effs (e.g., natural killer (NK) cells, natural killer T (NKT) cells, CD4⁺ effector T cells, and CD8⁺ effector T cells) and regulatory T cells (T_{Reg}S), limiting efficacy and resulting in harmful off-target effects and toxicities, most prominently severe vascular leak syndrome, which can lead to edema, organ failure, and death (Boyman et al., Nat Rev Immunol. 12(3):180-190 (2012); and Dhupkar et al., Adv Exp Med Biol. 995:33-51 (2017)). Furthermore, the vanishingly short serum half-life (<5 minutes) of IL-2 hinders its clinical performance (Donohue et al., J Immunol Baltim Md 1950. 130(5):2203-2208 (1983)).

### SUMMARY

IL-2 activates cell signaling through either a high-affinity (K_{D}≈pM) heterotrimeric receptor consisting of the IL-2Rα, IL-2Rβ, and γ_{c} chains, or an intermediate-affinity (K_{D}≈1 nM) heterodimeric receptor consisting of only the IL-2R13 and γ_{c} chains. Consequently, IL-2 responsiveness is determined by the IL-2Rα subunit, which is highly expressed on T_{Reg}s, but virtually absent from naïve Effs, rendering T_{Reg}s 100-fold more sensitive to IL-2 (see, e.g., Boyman et al., Nat Rev Immunol. 12(3):180-90 (2012); Malek, Annu Rev Immunol. 26:453-79 (2008); and Spangler et al., Annu Rev Immunol. 33:139-67 (2015)). The ability to isolate and selectively tune the immunostimulatory activities of IL-2 would represent a transformative advance for immunotherapeutic development, with important implications for treatment of cancer and infectious diseases.

This document provides methods and materials for targeted expansion of Effs. For example, provided herein are single-chain immunocytokines that can bind to an IL-2Rβ/γ_{c} polypeptide complex. In some cases, a single-chain immunocytokine that can bind to an IL-2Rβ/γ_{c} polypeptide complex can include (e.g., can be designed to include) an immunoglobulin heavy chain (V_{H}), an IL-2 polypeptide (or fragment thereof) that can bind an IL-2Rβ/γ_{c} polypeptide complex, and an immunoglobulin light chain (V_{L}). Also provided herein are methods for making and using single-chain immunocytokines that can bind to an IL-2Rβ/γ_{c} polypeptide complex. For example, a composition containing one or more single-chain immunocytokines that can bind to an IL-2Rβ/γ_{c} polypeptide complex can be administered to a mammal in need thereof (e.g., a mammal having a condition that can benefit from activating an immune response within the mammal such as a cancer and/or an infectious disease) to treat the mammal. In some cases, a composition containing one or more single-chain immunocytokines that can bind to an IL-2Rβ/γ_{c} polypeptide complex can be administered to a mammal to stimulate one or more Effs within the mammal (e.g., to activate an immune response in that mammal). For example, a composition containing one or more single-chain immunocytokines that can bind to an IL-2Rβ/γ_{c} polypeptide complex can be administered to a mammal having a cancer to treat the mammal. For example, a composition containing one or more single-chain immunocytokines that can bind to an IL-2Rβ/γ_{c} polypeptide complex can be administered to a mammal having, or at risk of developing, an infectious disease to treat the mammal.

As demonstrated herein, a single-chain immunocytokine engineered to bind to an IL-2Rβ/γ_{c} polypeptide complex can specifically stimulate (e.g., expand) immune Effs in vivo, and can inhibit tumor growth in vivo. Having the ability to stimulate immune Effs (e.g., but not T_{Reg}s) directly in a mammal (e.g., a human) provides unique and unrealized targeted cytokine therapies that can safely and selectively promote an effective immune response in a mammal (e.g., a human), and can be used to treat a mammal having, or suspected of having, a cancer and/or an infectious disease.

In general, one aspect of this document features single-chain immunocytokines including (a) an immunoglobulin heavy chain; (b) an IL-2 polypeptide, where the IL-2 polypeptide can bind an IL-2Rβ/γ_{c} polypeptide complex; and (c) an immunoglobulin light chain; where the single-chain immunocytokine binds to the IL-2Rβ/γ_{c} polypeptide complex. The immunoglobulin heavy chain can include a variable domain having at least 80% identity to an amino acid sequence set forth in SEQ ID NO:4 or having at least 80% identity to an amino acid sequence set forth in SEQ ID NO:28. The immunoglobulin heavy chain can include a variable domain having an amino acid sequence set forth in SEQ ID NO:4 or SEQ ID NO:28. The immunoglobulin heavy chain can include a γ heavy chain constant domain. The γ heavy chain constant domain can have at least 70% identity an amino acid sequence set forth in SEQ ID NO:5. The immunoglobulin heavy chain can include a constant domain having an amino acid sequence set forth in SEQ ID NO:5. The immunoglobulin heavy chain can include a signal sequence. The signal sequence can include an amino acid sequence set forth in SEQ ID NO:6. The immunoglobulin heavy chain can include an amino acid sequence set forth in SEQ ID NO:1 or SEQ ID NO:26. The IL-2 polypeptide can include an amino acid sequence having at least 80% identity to an amino acid sequence set forth in SEQ ID NO:9. The IL-2 polypeptide can include an amino acid sequence set forth in SEQ ID NO:9. The single-chain immunocytokine of claim 1, wherein said immunoglobulin light chain comprises a variable domain having at least 80% identity to an amino acid sequence set forth in SEQ ID NO: 10 or having at least 80% identity to an amino acid sequence set forth in SEQ ID NO:29. The immunoglobulin light chain can include a variable domain having an amino acid sequence set forth in SEQ ID NO:10 or SEQ ID NO:29. The immunoglobulin light chain can include a kappa (κ) light chain constant domain. The κ light chain constant domain can have at least 70% identity an amino acid sequence set forth in SEQ ID NO:11. The immunoglobulin light chain can include a constant domain having an amino acid sequence set forth in SEQ ID NO:11. The immunoglobulin light chain can include a signal sequence. The signal sequence can include an amino acid sequence set forth in SEQ ID NO:7. The immunoglobulin light chain can include an amino acid sequence set forth in SEQ ID NO:2. The IL-2 polypeptide and the immunoglobulin light chain can be a fusion polypeptide. The IL-2 polypeptide can include an amino acid sequence having at least 80% identity to an amino acid sequence set forth in SEQ ID NO:9. The IL-2 polypeptide can include an amino acid sequence set forth in SEQ ID NO:9. The immunoglobulin light chain can include a variable domain having at least 80% identity to an amino acid sequence set forth in SEQ ID NO: 10 or having at least 80% identity to an amino acid sequence set forth in SEQ ID NO:29. The immunoglobulin light chain can include a variable domain having an amino acid sequence set forth in SEQ ID NO:10 or SEQ ID NO:29. The immunoglobulin light chain can include a κ light chain constant domain. The κ light chain constant domain can have at least 70% identity an amino acid sequence set forth in SEQ ID NO:11. The immunoglobulin light chain can include a variable domain having an amino acid sequence set forth in SEQ ID NO:11. The IL-2 polypeptide and the immunoglobulin light chain can be fused via a linker. The linker can be a peptide linker that can include from 10 to 60 amino acids. The linker can be a (Gly₄Ser)₂ linker. The immunoglobulin light chain can include a signal sequence. The signal sequence can include an amino acid sequence set forth in SEQ ID NO:8. The immunoglobulin light chain can include an amino acid sequence set forth in SEQ ID NO:3, SEQ ID NO:23, SEQ ID NO:24, SEQ ID NO:25, or SEQ ID NO:27. The single-chain immunocytokine can have a half-life of from about 5 minutes to about 6 months. The single-chain immunocytokine can have an affinity for an IL-2Rβ polypeptide of from about 300 nM K_{D} to about 1 pM K_{D}. The single-chain immunocytokine can have an affinity for an IL-2Rα polypeptide of greater than about 10 nM K_{D}. In some cases, the single-chain immunocytokine can bind to a human IL-2Rβ/γ_{c} polypeptide complex. In some cases, the single-chain immunocytokine does not bind to a non-human IL-2Rβ/γ_{c} polypeptide complex.

In another aspect, this document features nucleic acid encoding a single-chain immunocytokine including (a) an immunoglobulin heavy chain; (b) an IL-2 polypeptide, where the IL-2 polypeptide can bind an IL-2Rβ/γ_{c} polypeptide complex; and (c) an immunoglobulin light chain; where the single-chain immunocytokine binds to the IL-2Rβ/γ_{c} polypeptide complex. The nucleic acid can include a first nucleic acid and a second nucleic acid, where the first nucleic acid can encode the immunoglobulin heavy chain, and where the second nucleic acid can encode the IL-2 polypeptide fused to the immunoglobulin light chain.

In another aspect, this document features methods for treating a mammal having cancer. The methods can include, or consist essentially of, administering a composition including one or more single-chain immunocytokines single-chain immunocytokines including (a) an immunoglobulin heavy chain; (b) an IL-2 polypeptide, where the IL-2 polypeptide can bind an IL-2Rβ/γ_{c} polypeptide complex; and (c) an immunoglobulin light chain; where the single-chain immunocytokine binds to the IL-2Rβ/γ_{c} polypeptide complex, or a composition including nucleic acid encoding a single-chain immunocytokine including (a) an immunoglobulin heavy chain; (b) an IL-2 polypeptide, where the IL-2 polypeptide can bind an IL-2Rβ/γ_{c} polypeptide complex; and (c) an immunoglobulin light chain; where the single-chain immunocytokine binds to the IL-2Rβ/γ_{c} polypeptide complex to a mammal having cancer. The mammal can be a human. The cancer can be breast cancer, ovarian cancer, prostate cancer, brain cancer, skin cancer, kidney cancer, lung cancer, melanoma, oral cancer, bladder cancer, colorectal cancer, cervical cancer, esophageal cancer, or uterine cancer. The method also can include administering one or more cancer treatments to the mammal under conditions where number of cancer cells present in the mammal is reduced. The method does not substantially activate regulatory T cells.

In another aspect, this document features methods for stimulating effector cells in a mammal. The methods can include, or consist essentially of, administering a composition including one or more single-chain immunocytokines single-chain immunocytokines including (a) an immunoglobulin heavy chain; (b) an IL-2 polypeptide, where the IL-2 polypeptide can bind an IL-2Rβ/γ_{c} polypeptide complex; and (c) an immunoglobulin light chain; where the single-chain immunocytokine binds to the IL-2Rβ/γ_{c} polypeptide complex, or a composition including nucleic acid encoding a single-chain immunocytokine including (a) an immunoglobulin heavy chain; (b) an IL-2 polypeptide, where the IL-2 polypeptide can bind an IL-2Rβ/γ_{c} polypeptide complex; and (c) an immunoglobulin light chain; where the single-chain immunocytokine binds to the IL-2Rβ/γ_{c} polypeptide complex to a mammal. The mammal can be a human. The method does not substantially activate regulatory T cells.

In another aspect, this document features methods for treating a mammal having an infectious disease. The methods can include, or consist essentially of, administering a composition including one or more single-chain immunocytokines single-chain immunocytokines including (a) an immunoglobulin heavy chain; (b) an IL-2 polypeptide, where the IL-2 polypeptide can bind an IL-2Rβ/γ_{c} polypeptide complex; and (c) an immunoglobulin light chain; where the single-chain immunocytokine binds to the IL-2Rβ/γ_{c} polypeptide complex, or a composition including nucleic acid encoding a single-chain immunocytokine including (a) an immunoglobulin heavy chain; (b) an IL-2 polypeptide, where the IL-2 polypeptide can bind an IL-2Rβ/γ_{c} polypeptide complex; and (c) an immunoglobulin light chain; where the single-chain immunocytokine binds to the IL-2Rβ/γ_{c} polypeptide complex to a mammal having an infectious disease. The mammal can be a human. The infectious disease can be human immunodeficiency virus, malaria, influenza, Ebola, tuberculosis, measles, rabies, Dengue fever, salmonellosis, whooping cough, plague, or West Nile fever. The method does not substantially activate regulatory T cells.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1C. Cytokine-antibody fusion (immunocytokine, IC) expression. FIG. 1A contains a schematic of the IL-2-602 immunocytokine layout. The IL-2 cytokine is fused to the N-terminal end of the light chain (LC) of the full-length 602 antibody, connected by a flexible linker. FIG. 1B contains a SDS-PAGE analysis of small-scale expression titrations of an IL-2-antibody immunocytokine. DNA transfection ratios of HC to IL-2-complexed LC are indicated. Bands are observed at the expected molecular weights (MW). FIG. 1C contains a size-exclusion chromatography trace from FPLC purification of an immunocytokine. SDS-PAGE analysis shows the protein was purified to homogeneity.

FIG. 2. IC exhibits biased IL-2 receptor binding. Binding titrations of hIL-2 and 602 IC to the immobilized human IL-2Rα or IL-2Rβ receptor subunits, as measured by bio-layer interferometry.

FIG. 3. 602 IC recapitulates signaling behavior of cytokine/antibody complex. STAT5 activation in response to IL-2, IL-2/602 complex, or 602 IC on YT-1 cells with (top) or without (bottom) IL-2Ra, as measured by flow cytometry.

FIGS. 4A-4B. IL-2/602 complex stimulates Eff activity. FIG. 4A contains a graph showing relative expansion of memory phenotype (MP) CD8⁺ effector T cells in spleens of mice treated with PBS, IL-2/602 complex, or IL-2/S4B6 daily for 4 days. FIG. 4B contains images of extracted Bl6F10 melanoma tumors from mice on day 20 post-inoculation. Mice were treated twice weekly with PBS or IL-2/602 complex.

FIGS. 5A-5B. Expression of 602 ICs with various linker lengths. FIG. 5A shows size exclusion chromatography traces from recombinantly produced 602 IC with linker lengths of 10, 15, 25, and 35 amino acids. Note that the earlier peaks (Peaks 1 and 2) correspond to oligomeric ICs whereas Peak 3 corresponds to the monomeric IC. FIG. 5B shows non-reducing and reducing SDS-PAGE analyses of recombinantly expressed 602 antibody (Ab), 602 IC LN15, 602 IC LN25, and 602 IC LN35.

FIG. 6. Linker length variant ICs exhibit expected IL-2 receptor binding properties. FIG. 6 shows binding titrations of IL-2, the 602 antibody (Ab), the IL-2+602 Ab complex (2:1 ratio, preincubated for 30 minutes at 37° C.), 602 IC variants, and an unrelated negative control protein to immobilized hIL-2Rβ, as measured by bio-layer interferometry.

FIGS. 7A-7C. 602 ICs exhibit biased signaling that favors immune effector cells. FIG. 7A shows STAT5 activation in response to IL-2, IL-2+602 antibody (Ab) complex (1:1 ratio, preincubated for 30 minutes at room temperature), or 602 IC variants on YT-1 human NK cells that express IL-2Rα (representative of T_{Reg} cells), as measured by flow cytometry. FIG. 7B shows STAT5 activation in response to IL-2, IL-2+602 Ab complex (1:1 ratio, preincubated for 30 minutes at room temperature), or 602 IC variants on YT-1 human NK cells that do not express IL-2Rα (a surrogate for immune effector cells), as measured by flow cytometry. FIG. 7C is a bar graph depicting the ratio of signaling potency on IL-2Rα− versus IL-2Rα+ YT-1 human NK cells for IL-2, IL-2+602 Ab, and 602 IC variants.

FIGS. 8A-8C. Engineered 602 IC variant shows improved binding to IL-2 and competition with IL-2Rα. FIG. 8A shows a flow cytometry-based yeast surface titration of soluble IL-2 to the yeast-displayed 602 single-chain variable fragment (scFv) or the post-round 5 evolved error-prone library (EP602) based on the 602 scFv. FIG. 8B shows a flow cytometry-based competition study wherein a saturated concentration of soluble IL-2 (10 nM) and the indicated concentrations of IL-2Rα were co-incubated with yeast cells displaying either the 602 scFv or the post-round 5 evolved error-prone library (EP602) based on the 602 scFv. FIG. 8C shows a flow cytometry-based competition study wherein a saturated concentration of soluble IL-2 (5 nM) and the indicated concentrations of IL-2Rα were co-incubated with yeast cells displaying either the 602 scFv or the evolved F10 scFv (a variant of the 602 scFv).

FIGS. 9A-9B. Expression of IC variants. FIG. 9A shows size exclusion chromatography traces from recombinantly produced 602 IC LN35, the 602 IC variant denoted F10 IC LN35, and a fusion protein comprising IL-2 linked to an irrelevant antibody (Irrel. Ab IC LN35). FIG. 9B shows non-reducing and reducing SDS-PAGE analyses of recombinantly expressed 602 IC LN35, F10 IC LN35, and Irrel. Ab IC LN35.

FIGS. 10A-10C. Engineered 602 IC variant exhibits expected IL-2 cytokine and receptor binding properties. FIG. 10A shows binding titrations of IL-2, the 602 antibody (Ab), the IL-2+602 Ab complex (2:1 ratio, preincubated for 30 minutes at 37° C.), 602 IC variants, and an unrelated negative control protein to immobilized hIL-2, as measured by bio-layer interferometry. FIG. 10B shows binding titrations of IL-2, the 602 Ab, the IL-2+602 Ab complex (2:1 ratio, preincubated for 30 minutes at 37° C.), 602 IC variants, and an unrelated negative control protein to immobilized hIL-2Rα, as measured by bio-layer interferometry. FIG. 10C shows binding titrations of IL-2, the 602 Ab, the IL-2+602 Ab complex (2:1 ratio, preincubated for 30 minutes at 37° C.), 602 IC variants, and an unrelated negative control to immobilized hIL-2Rβ, as measured by bio-layer interferometry.

FIGS. 11A-11C. Engineered 602 IC variant exhibits superior bias toward immune effector cells compared to parent IC. FIG. 11A shows STAT5 activation in response to IL-2, a fusion protein comprising IL-2 linked to an irrelevant antibody (Irrel. Ab IC LN35), IL-2+602 antibody (Ab) complex (1:1 ratio, preincubated for 30 minutes at room temperature), 602 IC LN35, or the 602 IC variant F10 IC LN35 on YT-1 human NK cells that express IL-2Ra (representative of T_{Reg} cells), as measured by flow cytometry. FIG. 11B STAT5 activation in response to IL-2, Irrel. Ab IC LN35, IL-2+602 Ab complex (1:1 ratio, preincubated for 30 minutes at room temperature), 602 IC LN35, or F10 IC LN35 on YT-1 human NK cells that do not express IL-2Rα (a surrogate for immune effector cells), as measured by flow cytometry. FIG. 11C is a bar graph depicting the ratio of signaling potency on IL-2Rα− versus IL-2Rα+ YT-1 human NK cells for IL-2, Irrel. Ab IC LN35, IL-2+602 Ab, 602 IC LN35, and F10 IC LN35

FIG. 12. A sequence (SEQ ID NO:1) of an exemplary recombinant antibody heavy chain that includes a signal sequence (bold), a 602 V_{H} (italic), and a mouse IgG2a C_{H}1, C_{H}2, and C_{H}3 ( [symbol] ).

FIG. 13. A sequence (SEQ ID NO:2) of an exemplary recombinant antibody light chain that includes a signal sequence (bold), a 602 V_{L} (italic), and a Kappa C_{L} ( [symbol] ).

FIG. 14. A sequence (SEQ ID NO:3) of an exemplary immunocytokine light chain (corresponding to 602 IC LN10) that includes a signal sequence (bold), human IL-2 (plain text), a linker (underlined), a 602 V_{L} (italic), and a Kappa C_{L} ( [symbol] ).

FIG. 15. A sequence (SEQ ID NO:23) of an exemplary immunocytokine light chain (corresponding to 602 IC LN15) that includes a signal sequence (bold), human IL-2 (plain text), a linker (underlined), a 602 V_{L} (italic), and a mouse Kappa C_{L} ( [symbol] ).

FIG. 16. A sequence (SEQ ID NO:24) of an exemplary immunocytokine light chain (corresponding to 602 IC LN25) that includes a signal sequence (bold), human IL-2 (plain text), a linker (underlined), a 602 V_{L} (italic), and a mouse Kappa C_{L} ( [symbol] ).

FIG. 17. A sequence (SEQ ID NO:25) of an exemplary immunocytokine light chain (corresponding to 602 IC LN35) that includes a signal sequence (bold), human IL-2 (plain text), a linker (underlined), a 602 V_{L} (italic), and a mouse Kappa C_{L} ( [symbol] ).

FIG. 18. A sequence (SEQ ID NO:26) of an exemplary immunocytokine heavy chain (corresponding to F10 IC LN35) that includes a signal sequence (bold), a 602 V_{H} (italic), and a mouse IgG2a C_{H}1, C_{H}2, and C_{H}3 ( [symbol] ). The mutation relative to 602 V_{H} is highlighted.

FIG. 19. A sequence (SEQ ID NO:27) of an exemplary immunocytokine light chain (corresponding to F10 IC LN35) that includes a signal sequence (bold), human IL-2 (plain text), a linker (underlined), a 602 V_{L} (italic), and a mouse Kappa C_{L} (bold and italic). Mutations relative to 602 V_{L} are highlighted.

### DETAILED DESCRIPTION

This document provides methods and materials for targeted expansion of Effs. For example, provided herein are single-chain immunocytokines that can bind to an IL-2Rβ/γ_{c} polypeptide complex. In some cases, a single-chain immunocytokine that can bind to an IL-2Rβ/γ_{c} polypeptide complex can include (e.g., can be designed to include) an immunoglobulin heavy chain, an IL-2 polypeptide (or fragment thereof) that can bind an IL-2Rβ/γ_{c} polypeptide complex, and an immunoglobulin light chain. Also provided herein are methods for making and using single-chain immunocytokines that can bind to an IL-2Rβ/γ_{c} polypeptide complex. For example, a composition containing one or more single-chain immunocytokines that can bind to an IL-2Rβ/γ_{c} polypeptide complex can be administered to a mammal (e.g., a human) in need thereof (e.g., a mammal having a condition that can benefit from activating an immune response within the mammal such as a cancer and/or an infectious disease) to treat the mammal. In some cases, a composition containing one or more single-chain immunocytokines that can bind to an IL-2Rβ/γ_{c} polypeptide complex can be administered to a mammal to stimulate Effs within the mammal (e.g., to activate an immune response in that mammal). Examples of Effs that can be stimulated by a single-chain immunocytokine that can bind to an IL-2Rβ polypeptide described herein include, without limitation, CD4⁺ effector T cells, CD8⁺ effector T cells, memory phenotype CD8⁺ effector T cell, NK cells, and NKT cells. For example, a composition containing one or more single-chain immunocytokines that can bind to an IL-2Rβ/γ_{c} polypeptide complex can be administered to a mammal having a cancer to treat the mammal. For example, a composition containing one or more single-chain immunocytokines that can bind to an IL-2Rβ/γ_{c} polypeptide complex can be administered to a mammal having, or at risk of developing, an infectious disease to treat the mammal.

As used herein, a single-chain immunocytokine described herein (e.g., a single-chain immunocytokine that can bind an IL-2Rβ/γ_{c} polypeptide complex) is a fusion protein that includes a cytokine fused (e.g., genetically fused) to antibody or a fragment thereof (e.g., a cytokine/antibody fusion protein). In some cases, a single-chain immunocytokine described herein can include a cytokine fused to an anti-cytokine antibody or a fragment thereof (e.g., an anti-IL-2 antibody or a fragment thereof). In some cases, a single-chain immunocytokine described herein can include a cytokine that is fused to an antibody such that the cytokine and antibody bind intramolecularly within the immunocytokine. In some cases, a single-chain immunocytokine described herein can include a cytokine that is fused to one or more ends of an antibody (e.g., the N- or C-terminus of an antibody heavy chain and/or the N- or C-terminus of an antibody light chain). For example, a single-chain immunocytokine described herein can be a fusion polypeptide that includes an immunoglobulin heavy chain (e.g., an immunoglobulin heavy chain from anti-cytokine antibody) fused to an IL-2 polypeptide (or fragment thereof) that can bind an IL-2Rβ/γ_{c} polypeptide complex fused to an immunoglobulin light chain (e.g., an immunoglobulin light chain from anti-cytokine antibody).

A single-chain immunocytokine described herein (e.g., a single-chain immunocytokine that can bind to an IL-2Rβ/γ_{c} polypeptide complex) can bind to an IL-IL-2Rβ/γ_{c} polypeptide complex from any appropriate source (e.g., from any appropriate mammal such as a human or a mouse). In some cases, IL-2 polypeptide (or fragment thereof) that can bind an IL-2Rβ/γ_{c} polypeptide complex can bind to a human IL-2Rβ/γ_{c} polypeptide complex. In some cases, where an IL-2 polypeptide (or fragment thereof) that can bind an IL-2Rβ/γ_{c} polypeptide complex binds to an IL-2Rβ/γ_{c} polypeptide complex from a first species of mammal, the IL-2 polypeptide (or fragment thereof) that can bind an IL-2Rβ/γ_{c} polypeptide complex does not cross-react with an IL-2Rβ/γ_{c} polypeptide complex from a second species of mammal. For example, when an IL-2 polypeptide (or fragment thereof) that can bind an IL-2Rβ/γ_{c} polypeptide complex binds to a human IL-2Rβ/γ_{c} polypeptide complex, the IL-2 polypeptide (or fragment thereof) that can bind an IL-2Rβ/γ_{c} polypeptide complex does not cross-react with an IL-2Rβ/γ_{c} polypeptide complex from a non-human species (e.g., a mouse IL-2Rβ/γ_{c} polypeptide complex).

A single-chain immunocytokine described herein (e.g., a single-chain immunocytokine that can bind to an IL-2Rβ/γ_{c} polypeptide complex) can include any appropriate immunoglobulin (Ig) heavy chain. An immunoglobulin heavy chain can be from any appropriate isotype immunoglobulin (e.g., a IgA immunoglobulin, a IgD immunoglobulin, a IgE immunoglobulin, a IgG immunoglobulin, and a IgM immunoglobulin). In some cases, an immunoglobulin heavy chain can be an IgG heavy chain (e.g., an IgG2a heavy chain). An immunoglobulin heavy chain can be from any appropriate class of immunoglobulin (e.g., γ, σ, α, μ, and ε). An immunoglobulin heavy chain can have any appropriate heavy chain variable domain (V_{H}). An immunoglobulin heavy chain can have any appropriate heavy chain constant domains (C_{H}). In some cases, an immunoglobulin heavy chain can be an immunoglobulin having three constant domains (e.g., C_{H}1, C_{H}2, and C_{H}3) such as a γ heavy chain, an α heavy chain, or a δ heavy chain. In some cases, an immunoglobulin heavy chain can be an immunoglobulin having four constant domains (e.g., C_{H}1, C_{H}2, C_{H}3, and C_{H}4) such as a μ heavy chain or a ε heavy chain. An immunoglobulin heavy chain can be from any appropriate immunoglobulin. In some cases, the immunoglobulin heavy chain variable domain and the immunoglobulin heavy chain constant domains can be from the same immunoglobulin. In some cases, the immunoglobulin heavy chain variable domain and the immunoglobulin heavy chain constant domains can be from different immunoglobulins. In some cases, the immunoglobulin heavy chain variable domain and/or the immunoglobulin heavy chain constant domains can be from a naturally occurring immunoglobulin (e.g., can be derived from a naturally occurring immunoglobulin). In some cases, the immunoglobulin heavy chain variable domain and/or the immunoglobulin heavy chain constant domains can be synthetic. Examples of immunoglobulins whose heavy chain variable domain and/or the immunoglobulin heavy chain constant domains can be used in a single-chain immunocytokine described herein include, without limitation, monoclonal antibody 602 (MAB602, referred to herein as “602”) heavy chains (see, e.g., R&D systems #MAB602-SP). In some cases, immunoglobulins whose heavy chain variable domains and/or heavy chain constant domains can be used in a single-chain immunocytokine described herein can be as described elsewhere (see, e.g., Krieg et al., Proc Natl Acad Sci USA. 107(26):11906-11 (2010)). An immunoglobulin heavy chain can include any appropriate sequence (e.g., amino acid sequence). In some cases, an immunoglobulin heavy chain variable domain can include an amino acid sequence having at least about 80% identity (e.g., about 82%, about 85%, about 88%, about 90%, about 93%, about 95%, about 97%, about, 98%, about 99%, or 100% sequence identity) to the amino acid sequence set forth in SEQ ID NO:4. For example, a single-chain immunocytokine described herein can include an immunoglobulin heavy chain variable domain having the amino acid sequence set forth in SEQ ID NO:4. In some cases, an immunoglobulin heavy chain variable domain can include an amino acid sequence having at least about 80% identity (e.g., about 82%, about 85%, about 88%, about 90%, about 93%, about 95%, about 97%, about, 98%, about 99%, or 100% sequence identity) to the amino acid sequence set forth in SEQ ID NO:28. For example, a single-chain immunocytokine described herein can include an immunoglobulin heavy chain variable domain having the amino acid sequence set forth in SEQ ID NO:28. In some cases, an immunoglobulin heavy chain constant domain can include an amino acid sequence having at least about 70% identity (e.g., about 75%, about 80%, about 85%, about 88%, about 90%, about 93%, about 95%, about 97%, about, 98%, about 99%, or 100% sequence identity) to the amino acid sequence set forth in SEQ ID NO:5. For example, a single-chain immunocytokine described herein can include an immunoglobulin heavy chain constant domain having the amino acid sequence set forth in SEQ ID NO:5. In some cases, an immunoglobulin heavy chain also can include a signal sequence. A signal sequence can be any appropriate signal sequence (e.g., (SEQ ID NO:6 and SEQ ID NO:7). For example, a single-chain immunocytokine described herein can include an immunoglobulin heavy chain having a signal sequence with the amino acid sequence set forth in SEQ ID NO:6.

An exemplary immunoglobulin heavy chain that can be used in a single-chain immunocytokine described herein (e.g., a single-chain immunocytokine that can bind to an IL-2Rβ/γ_{c} polypeptide complex) can be as set forth in SEQ ID NO:1 or SEQ ID NO:26. For example, an immunoglobulin heavy chain that can be used in a single-chain immunocytokine described herein can include a signal sequence, a variable domain from a 602 antibody, and an IgG2a constant domain (e.g., a mouse IgG2a constant domain). In some cases, an immunoglobulin heavy chain that can be used in a single-chain immunocytokine described herein can include a signal sequence having the amino acid sequence set forth in SEQ ID NO:6, a variable domain having the amino acid sequence set forth in SEQ ID NO:4, and a constant domain having the amino acid sequence set forth in SEQ ID NO:5. For example, an immunoglobulin heavy chain that can be used in a single-chain immunocytokine described herein can include the amino acid sequence set forth in SEQ ID NO:1. In some cases, an immunoglobulin heavy chain that can be used in a single-chain immunocytokine described herein can include a signal sequence having the amino acid sequence set forth in SEQ ID NO:6, a variable domain having the amino acid sequence set forth in SEQ ID NO:28, and a constant domain having the amino acid sequence set forth in SEQ ID NO:5. For example, an immunoglobulin heavy chain that can be used in a single-chain immunocytokine described herein can include the amino acid sequence set forth in SEQ ID NO:26. In some cases, an immunoglobulin heavy chain can have one or more modifications to the amino acid sequence (e.g., one or more modifications to SEQ ID NO:1 or one or more modifications to SEQ ID NO:26). In some cases, a modification to the amino acid sequence of α heavy chain included in a single-chain immunocytokine described herein can alter the cytokine affinity of the single-chain immunocytokine. In some cases, a modification to the amino acid sequence of a heavy chain included in a single-chain immunocytokine described herein can alter the receptor competition (e.g., can alter the binding properties) of the single-chain immunocytokine.

A single-chain immunocytokine described herein (e.g., a single-chain immunocytokine that can bind to an IL-2Rβ/γ_{c} polypeptide complex) can include any appropriate IL-2 polypeptide (or fragment thereof) that can bind an IL-2Rβ/γ_{c} polypeptide complex. An IL-2 polypeptide (or fragment thereof) that can bind an IL-2Rβ/γ_{c} polypeptide complex can be from any source. In some cases, an IL-2 polypeptide (or fragment thereof) that can bind an IL-2Rβ/γ_{c} polypeptide complex can be a naturally occurring IL-2 polypeptide (or fragment thereof) that can bind an IL-2Rβ/γ_{c} polypeptide complex. In some cases, an IL-2 polypeptide (or fragment thereof) that can bind an IL-2Rβ/γ_{c} polypeptide complex can be synthetic. An IL-2 polypeptide (or fragment thereof) that can bind an IL-2Rβ/γ_{c} polypeptide complex can have any appropriate sequence. In some cases, an IL-2 polypeptide (or fragment thereof) that can bind an IL-2Rβ/γ_{c} polypeptide complex can include an amino acid sequence having at least about 80% identity (e.g., about 82%, about 85%, about 88%, about 90%, about 93%, about 95%, about 97%, about, 98%, about 99%, or 100% sequence identity) to the amino acid sequence set forth in SEQ ID NO:9. For example, a single-chain immunocytokine described herein can include an immunoglobulin heavy chain constant domain having the amino acid sequence set forth in SEQ ID NO:9. In some cases, an IL-2 polypeptide (or fragment thereof) that can bind an IL-2Rβ/γ_{c} polypeptide complex can have one or more modifications to the amino acid sequence (e.g., one or more modifications to SEQ ID NO:9). In some cases, a modification to the amino acid sequence of IL-2 polypeptide (or fragment thereof) that can bind an IL-2Rβ/γ_{c} polypeptide complex included in a single-chain immunocytokine described herein can mitigate disruption of the intramolecular assembly of the single-chain immunocytokine. In some cases, a modification to the amino acid sequence of α heavy chain included in a single-chain immunocytokine described herein can enhance the activity (e.g., signaling activity) of the single-chain immunocytokine.

A single-chain immunocytokine described herein (e.g., a single-chain immunocytokine that can bind to an IL-2Rβ/γ_{c} polypeptide complex) can include any appropriate immunoglobulin light chain. An immunoglobulin light chain can be from any appropriate type of immunoglobulin light chain (e.g., a (κ) light chain and a lambda (λ) light chain). In some cases, an immunoglobulin light chain can be a κ light chain. An immunoglobulin light chain can have any appropriate light chain variable domain (V_{L}). An immunoglobulin light chain can have any appropriate light chain constant domain (C_{L}). An immunoglobulin light chain can be from any appropriate immunoglobulin. In some cases, the immunoglobulin light chain variable domain and the immunoglobulin light chain constant domains can be from the same immunoglobulin. In some cases, the immunoglobulin light chain variable domain and the immunoglobulin light chain constant domains can be from different immunoglobulins. In some cases, the immunoglobulin light chain variable domain and/or the immunoglobulin light chain constant domains can be from a naturally occurring immunoglobulin (e.g., can be derived from a naturally occurring immunoglobulin). In some cases, the immunoglobulin light chain variable domain and/or the immunoglobulin light chain constant domains can be synthetic. Examples of immunoglobulins whose light chain variable domain and/or the immunoglobulin light chain constant domains can be used in a single-chain immunocytokine described herein include, without limitation, 602 light chains (see, e.g., R&D systems #MAB602-SP). In some cases, immunoglobulins whose light chain variable domains and/or light chain constant domains can be used in a single-chain immunocytokine described herein can be as described elsewhere (see, e.g., Krieg et al., Proc Natl Acad Sci USA. 107(26):11906-11 (2010)). An immunoglobulin heavy chain can include any appropriate sequence (e.g., amino acid sequence). An immunoglobulin light chain can include any appropriate sequence (e.g., amino acid sequence). In some cases, an immunoglobulin light chain variable domain can include an amino acid sequence having at least about 80% identity (e.g., about 82%, about 85%, about 88%, about 90%, about 93%, about 95%, about 97%, about, 98%, about 99%, or 100% sequence identity) to the amino acid sequence set forth in SEQ ID NO:10. For example, a single-chain immunocytokine described herein can include an immunoglobulin light chain variable domain having the amino acid sequence set forth in SEQ ID NO:10. In some cases, an immunoglobulin light chain variable domain can include an amino acid sequence having at least about 80% identity (e.g., about 82%, about 85%, about 88%, about 90%, about 93%, about 95%, about 97%, about, 98%, about 99%, or 100% sequence identity) to the amino acid sequence set forth in SEQ ID NO:29. For example, a single-chain immunocytokine described herein can include an immunoglobulin light chain variable domain having the amino acid sequence set forth in SEQ ID NO:29. In some cases, an immunoglobulin light chain constant domain can include an amino acid sequence having at least about 70% identity (e.g., about 75%, about 80%, about 85%, about 88%, about 90%, about 93%, about 95%, about 97%, about, 98%, about 99%, or 100% sequence identity) to the amino acid sequence set forth in SEQ ID NO:11. For example, a single-chain immunocytokine described herein can include an immunoglobulin light chain constant domain having the amino acid sequence set forth in SEQ ID NO:11. In some cases, an immunoglobulin light chain also can include a signal sequence. A signal sequence can be any appropriate signal sequence (e.g., SEQ ID NO:7 and SEQ ID NO:8). For example, a single-chain immunocytokine described herein can include an immunoglobulin light chain having a signal sequence with the amino acid sequence set forth in SEQ ID NO:7.

An exemplary immunoglobulin light chain that can be used in a single-chain immunocytokine described herein (e.g., a single-chain immunocytokine that can bind to an IL-2Rβ/γ_{c} polypeptide complex) can be as set forth in SEQ ID NO:2. For example, an immunoglobulin light chain that can be used in a single-chain immunocytokine described herein can include a signal sequence, a variable domain from a 602 antibody, and a κ constant domain. For example, an immunoglobulin light chain that can be used in a single-chain immunocytokine described herein can include a signal sequence having the amino acid sequence set forth in SEQ ID NO:7, a variable domain having the amino acid sequence set forth in SEQ ID NO:10, and a constant domain having the amino acid sequence set forth in SEQ ID NO:11. In some cases, an immunoglobulin light chain that can be used in a single-chain immunocytokine described herein can include the amino acid sequence set forth in SEQ ID NO:2. In some cases, an immunoglobulin light chain can have one or more modifications to the amino acid sequence (e.g., one or more modifications to SEQ ID NO:2). In some cases, a modification to the amino acid sequence of a light chain included in a single-chain immunocytokine described herein can alter the cytokine affinity of the single-chain immunocytokine. In some cases, a modification to the amino acid sequence of a light chain included in a single-chain immunocytokine described herein can alter the receptor competition (e.g., can alter the binding properties) of the single-chain immunocytokine.

In some cases, an immunoglobulin light chain can include an IL-2 polypeptide (or fragment thereof) that can bind an IL-2Rβ/γ_{c} polypeptide complex described herein. In cases where an immunoglobulin light chain includes the IL-2 polypeptide (or fragment thereof) that can bind an IL-2Rβ/γ_{c} polypeptide complex, the IL-2 polypeptide (or fragment thereof) that can bind an IL-2Rβ/γ_{c} polypeptide complex can be in any appropriate location within the immunoglobulin light chain. In some cases, the IL-2 polypeptide (or fragment thereof) that can bind an IL-2Rβ/γ_{c} polypeptide complex can be fused to the immunoglobulin light chain (e.g., the immunoglobulin light chain variable domain). When the IL-2 polypeptide (or fragment thereof) that can bind an IL-2Rβ/γ_{c} polypeptide complex and the immunoglobulin light chain variable domain are a fusion polypeptide, the IL-2 polypeptide (or fragment thereof) that can bind an IL-2Rβ/γ_{c} polypeptide complex and the immunoglobulin light chain variable domain can be fused via a linker. A linker can be any appropriate linker. In some cases, a linker can be flexible (e.g., to allow for intramolecular interaction(s)). In some cases, a linker can be a peptide linker. A peptide linker can include any appropriate number of amino acids. For example, a peptide linker can include from about 10 amino acids to about 60 amino acids (e.g., from about 10 amino acids to about 50 amino acids, from about 10 amino acids to about 40 amino acids, from about 10 amino acids to about 30 amino acids, from about 20 amino acids to about 60 amino acids, from about 30 amino acids to about 60 amino acids, from about 40 amino acids to about 60 amino acids, from about 50 amino acids to about 60 amino acids, from about 15 amino acids to about 55 amino acids, from about 20 amino acids to about 50 amino acids, from about 30 amino acids to about 40 amino acids, from about 20 amino acids to about 40 amino acids, from about 30 amino acids to about 50 amino acids, or from about 40 amino acids to about 60 amino acids). A peptide linker can include any appropriate amino acids. For example, a peptide linker can include one or more glycine (Gly) residues and/or one or more serine (Ser) residues. Examples of linkers that can be used to fuse an IL-2 polypeptide (or fragment thereof) that can bind an IL-2Rα/IL-2Rβ/γ_{c} polypeptide complex to an immunoglobulin light chain variable domain include, without limitation, a (Gly₄Ser)₂ linker (SEQ ID NO:12), a (Gly₄Ser)₃ linker (SEQ ID NO:13), a (Gly₄Ser)₄ linker (SEQ ID NO:14), a (Gly₄Ser)₅ linker (SEQ ID NO:15), a (Gly₄Ser)₆ linker (SEQ ID NO:16), (Gly₄Ser)₇ linker (SEQ ID NO:17), a (Gly₄Ser)₈ linker (SEQ ID NO:18), a (Gly₄Ser)₉ linker (SEQ ID NO:19), a (Gly₄Ser)₁₀ linker (SEQ ID NO:20), a (Gly₄Ser)₁₁ linker (SEQ ID NO:21), and a (Gly₄Ser)₁₂ linker (SEQ ID NO:22). For example, a single-chain immunocytokine described herein can include an immunoglobulin light chain having an IL-2 polypeptide (or fragment thereof) that can bind an IL-2Rβ/γ_{c} polypeptide complex fused to an immunoglobulin light chain variable domain via a linker having the amino acid sequence set forth in SEQ ID NO:12 or SEQ ID NO:13. In some cases, an IL-2 polypeptide (or fragment thereof) that can bind an IL-2Rβ/γ_{c} polypeptide complex can have one or more modifications to the amino acid sequence (e.g., one or more modifications to SEQ ID NO:12 or one or more modifications to SEQ ID NO:13). In some cases, a modification to the amino acid sequence of a linker can alter the length, charge, structure, and/or composition of the linker.

An exemplary immunoglobulin light chain that includes an IL-2 polypeptide (or fragment thereof) that can bind an IL-2Rβ/γ_{c} polypeptide complex that can be used in a single-chain immunocytokine described herein (e.g., a single-chain immunocytokine that can bind to an IL-2Rβ polypeptide) can be as set forth in any one of SEQ ID NO:3, SEQ ID NO:23, SEQ ID NO:24, SEQ ID NO:25, or SEQ ID NO:27. For example, an immunoglobulin light chain that can be used in a single-chain immunocytokine described herein can include a signal sequence, an IL-2 polypeptide (or fragment thereof) that can bind an IL-2Rβ/γ_{c} polypeptide complex, a linker, a variable domain from a 602 antibody, and a κ constant domain. For example, an immunoglobulin light chain that can be used in a single-chain immunocytokine described herein can include a signal sequence having the amino acid sequence set forth in SEQ ID NO:8, an IL-2 polypeptide having the amino acid sequence set forth in SEQ ID NO: 9, a linker having the amino acid sequence set forth in SEQ ID NO:12, a variable domain having the amino acid sequence set forth in SEQ ID NO:10, and a constant domain having the amino acid sequence set forth in SEQ ID NO:11. In some cases, an immunoglobulin light chain that can be used in a single-chain immunocytokine described herein can include the amino acid sequence set forth in SEQ ID NO:3. For example, an immunoglobulin light chain that can be used in a single-chain immunocytokine described herein can include a signal sequence having the amino acid sequence set forth in SEQ ID NO:8, an IL-2 polypeptide having the amino acid sequence set forth in SEQ ID NO: 9, a linker having the amino acid sequence set forth in SEQ ID NO:13, a variable domain having the amino acid sequence set forth in SEQ ID NO:10, and a constant domain having the amino acid sequence set forth in SEQ ID NO:11. In some cases, an immunoglobulin light chain that can be used in a single-chain immunocytokine described herein can include the amino acid sequence set forth in SEQ ID NO:23. For example, an immunoglobulin light chain that can be used in a single-chain immunocytokine described herein can include a signal sequence having the amino acid sequence set forth in SEQ ID NO:8, an IL-2 polypeptide having the amino acid sequence set forth in SEQ ID NO: 9, a linker having the amino acid sequence set forth in SEQ ID NO:15, a variable domain having the amino acid sequence set forth in SEQ ID NO:10, and a constant domain having the amino acid sequence set forth in SEQ ID NO:11. In some cases, an immunoglobulin light chain that can be used in a single-chain immunocytokine described herein can include the amino acid sequence set forth in SEQ ID NO:24. For example, an immunoglobulin light chain that can be used in a single-chain immunocytokine described herein can include a signal sequence having the amino acid sequence set forth in SEQ ID NO:8, an IL-2 polypeptide having the amino acid sequence set forth in SEQ ID NO: 9, a linker having the amino acid sequence set forth in SEQ ID NO:17, a variable domain having the amino acid sequence set forth in SEQ ID NO:10, and a constant domain having the amino acid sequence set forth in SEQ ID NO:11. In some cases, an immunoglobulin light chain that can be used in a single-chain immunocytokine described herein can include the amino acid sequence set forth in SEQ ID NO:25. For example, an immunoglobulin light chain that can be used in a single-chain immunocytokine described herein can include a signal sequence having the amino acid sequence set forth in SEQ ID NO:8, an IL-2 polypeptide having the amino acid sequence set forth in SEQ ID NO: 9, a linker having the amino acid sequence set forth in SEQ ID NO:17, a variable domain having the amino acid sequence set forth in SEQ ID NO:29, and a constant domain having the amino acid sequence set forth in SEQ ID NO:11. In some cases, an immunoglobulin light chain that can be used in a single-chain immunocytokine described herein can include the amino acid sequence set forth in SEQ ID NO:27. In some cases, an immunoglobulin light chain can have one or more modifications to the amino acid sequence (e.g., one or more modifications to SEQ ID NO:3, one or more modifications to SEQ ID NO:23, one or more modifications to SEQ ID NO:24, one or more modifications to SEQ ID NO:25, or one or more modifications to SEQ ID NO:27). In some cases, a modification to the amino acid sequence of a light chain included in a single-chain immunocytokine described herein can alter the cytokine affinity of the single-chain immunocytokine. In some cases, a modification to the amino acid sequence of a light chain included in a single-chain immunocytokine described herein can alter the receptor competition (e.g., can alter the binding properties) of the single-chain immunocytokine.

In some cases, a single-chain immunocytokine described herein (e.g., a single-chain immunocytokine that can bind to an IL-2Rβ/γ_{c} polypeptide complex) can be a stable molecule (e.g., as compared to a molecule that can bind to an IL-2Rβ/γ_{c} polypeptide complex that is not present in a single-chain immunocytokine described herein). For example, a single-chain immunocytokine described herein can have a half-life (e.g., an in vivo half-life such as a serum half-life or a plasma half-life) of from about 5 minutes to about 6 months (e.g., from about 15 minutes to about 6 months, from about 30 minutes to about 6 months, from about 1 hour to about 6 months, from about 24 hours to about 6 months, from about 3 days to about 6 months, from about 7 days to about 6 months, from about 1 month to about 6 months, from about 3 months to about 6 months, from about 5 minutes to about 3 months, from about 5 minutes to about 1 month, from about 5 minutes to about 2 weeks, from about 5 minutes to about 7 days, from about 5 minutes to about 3 days, from about 5 minutes to about 24 hours, from about 5 minutes to about 12 hours, from about 5 minutes to about 60 minutes, from about 30 minutes to about 3 days, from about 3 days to about 1 week, from about 1 week to about 1 month, or from about 1 month to about 3 months). For example, a single-chain immunocytokine described herein can have a shelf life at standard room temperature conditions (e.g., about 25° C.) for from about 1 day to about 1 month (e.g., from about 1 day to about 2 weeks, from about 1 day to about 1 week, from about 1 day to about 5 days, from about 4 days to about 1 month, from about 1 week to about 1 month, from about 2 weeks to about 1 month, from about 3 days to about 2 weeks, from about 2 days to about 5 days, from about 5 days to about 2 weeks, or from about 1 week to about 3 weeks). For example, thermal shift assay, protein stability curve analysis, size exclusion chromatography, and/or dynamic light scattering can be used to determine the stability of a single-chain immunocytokine described herein.

In some cases, a single-chain immunocytokine described herein (e.g., a single-chain immunocytokine that can bind to an IL-2Rβ/γ_{c} polypeptide complex) can have an enhanced interaction with (e.g., stronger binding affinity for) an IL-2Rβ polypeptide (e.g., as compared to a molecule that can bind to an IL-2Rβ polypeptide complex that is not present in a single-chain immunocytokine described herein). For example, a single-chain immunocytokine described herein can have an affinity for an IL-2Rβ/γ_{c} polypeptide complex of from about 300 nM K_{D} to about 1 pM K_{D}.

In some cases, a single-chain immunocytokine described herein (e.g., a single-chain immunocytokine that can bind to an IL-2Rβ/γ_{c} polypeptide complex) can have a reduced or eliminated interaction with (e.g., weaker binding affinity for) an IL-2Rα polypeptide (e.g., as compared to a molecule that can bind to an IL-2Rα polypeptide complex that is not present in a single-chain immunocytokine described herein). For example, a single-chain immunocytokine described herein can have an affinity for an IL-2Rα polypeptide of greater than about 10 nM K_{D}.

Any appropriate method can be used to determine the binding affinity between a single-chain immunocytokine described herein (e.g., a single-chain immunocytokine that can bind to an IL-2Rβ polypeptide) and an IL-2Rβ polypeptide and/or an IL-2Rα polypeptide. For example, affinity titration studies, surface plasmon resonance, isothermal calorimetry, and/or bio-layer interferometry can be used to determine the binding affinity between a single-chain immunocytokine described herein and an IL-2Rβ polypeptide and/or an IL-2Rα polypeptide.

In some cases, a single-chain immunocytokine described herein (e.g., a single-chain immunocytokine that can bind to an IL-2Rβ/γ_{c} polypeptide complex) can activate a reduced or eliminated number T_{Reg}s (e.g., as compared to a molecule that can bind to an IL-2Rβ/γ_{c} polypeptide complex that is not present in a single-chain immunocytokine described herein). For example, a single-chain immunocytokine described herein does not does not substantially activate T_{Reg}s (e.g., does not active T_{Reg}s to a detectable level and/or a level sufficient to suppress or downregulate activation of Effs). Any appropriate method can be used to determine the presence, absence, amount, or activity of T_{Reg}s. For example, immunostaining for T_{Reg} markers (e.g., CD4, IL-2Rα, and/or Foxp3) and/or assessing activity based on ELISA or signal transducer and activator of transcription (STAT) 5 phosphorylation can be used to determine the presence, absence, amount, or activity of T_{Reg}s.

This document also provides methods and materials for making single-chain immunocytokines described herein (e.g., a single-chain immunocytokine that can bind to an IL-2Rβ/γ_{c} polypeptide complex). For example, this document also provides nucleic acid (e.g., nucleic acid vectors) that can encode a polypeptide that can be used to generate single-chain immunocytokines described herein are provided. In some cases, nucleic acid can encode an immunoglobulin heavy chain, an IL-2 polypeptide (or fragment thereof) that can bind an IL-2Rβ/γ_{c} polypeptide complex, and/or an immunoglobulin light chain that can be used to generate a single-chain immunocytokine that can bind to an IL-2Rβ/γ_{c} polypeptide complex. For example, a first nucleic acid can encode an immunoglobulin heavy chain, and a second nucleic acid can encode an IL-2 polypeptide (or fragment thereof) that can bind an IL-2Rβ/γ_{c} polypeptide complex fused to an immunoglobulin light chain.

Nucleic acid (e.g., nucleic acid vectors) encoding one or more polypeptides (e.g., an immunoglobulin heavy chain, an IL-2 polypeptide (or fragment thereof) that can bind an IL-2Rβ/γ_{c} polypeptide complex, and/or an immunoglobulin light chain) that can be used to generate polypeptide that can be used to generate single-chain immunocytokines described herein (e.g., a single-chain immunocytokine that can bind to an IL-2Rβ/γ_{c} polypeptide complex) can be any appropriate nucleic acid. Nucleic acid can be DNA (e.g., a DNA construct), RNA (e.g., mRNA), or a combination thereof. In some cases, nucleic acid encoding one or more polypeptides that can be used to generate polypeptide that can be used to generate single-chain immunocytokines described herein can be a vector (e.g., an expression vector or a plasmid).

In some cases, nucleic acid encoding one or more polypeptides (e.g., an immunoglobulin heavy chain, an IL-2 polypeptide (or fragment thereof) that can bind an IL-2Rβ/γ_{c} polypeptide complex, and/or an immunoglobulin light chain) that can be used to generate polypeptide that can be used to generate single-chain immunocytokines described herein (e.g., a single-chain immunocytokine that can bind to an IL-2Rβ/γ_{c} polypeptide complex) also can include one or more regulatory elements (e.g., to regulate expression of the amino acid chain). Examples of regulatory elements that can be included in nucleic acid encoding one or more polypeptides that can be used to generate polypeptide that can be used to generate single-chain immunocytokines described herein include, without limitation, promoters (e.g., constitutive promoters, tissue/cell-specific promoters, and inducible promoters such as chemically-activated promoters and light-activated promoters), and enhancers.

In some cases, one or more polypeptides (e.g., an immunoglobulin heavy chain, an IL-2 polypeptide (or fragment thereof) that can bind an IL-2Rβ/γ_{c} polypeptide complex, and/or an immunoglobulin light chain) encoded by nucleic acid described herein can be used to generate single-chain immunocytokines described herein (e.g., a single-chain immunocytokine that can bind to an IL-2Rβ/γ_{c} polypeptide complex). For example, two or more polypeptides including an immunoglobulin heavy chain, an IL-2 polypeptide (or fragment thereof) that can bind an IL-2Rβ/γ_{c} polypeptide complex, and an immunoglobulin light chain can assemble (e.g., can self-assemble) into a single-chain immunocytokine described herein (e.g., a single-chain immunocytokine that can bind to an IL-2Rβ/γ_{c} polypeptide complex). In some cases, an immunoglobulin heavy chain encoded by a first nucleic acid, and an IL-2 polypeptide (or fragment thereof) that can bind an IL-2Rβ/γ_{c} polypeptide complex fused to an immunoglobulin light chain encoded by a second nucleic acid can assemble (e.g., can self-assemble) into a single-chain immunocytokine described herein. When two or more polypeptides including an immunoglobulin heavy chain, an IL-2 polypeptide (or fragment thereof) that can bind an IL-2Rβ/γ_{c} polypeptide complex, and an immunoglobulin light chain assemble into a single-chain immunocytokine described herein, the two or more polypeptides can assemble in vivo or in vitro.

In some cases, single-chain immunocytokines described herein (e.g., a single-chain immunocytokine that can bind to an IL-2Rβ/γ_{c} polypeptide complex), or nucleic acid encoding one or more polypeptides (e.g., an immunoglobulin heavy chain, an IL-2 polypeptide (or fragment thereof) that can bind an IL-2Rβ/γ_{c} polypeptide complex, and/or an immunoglobulin light chain) that can be used to generate polypeptide that can be used to generate single-chain immunocytokines described herein, can be purified. A “purified” polypeptide or nucleic acid refers to a polypeptide or nucleic acid that constitutes the major component in a mixture of components, e.g., 30% or more, 40% or more, 50% or more, 60% or more, 70% or more, 80% or more, 90% or more, 95% or more, or 99% or more by weight. For example, a purified single-chain immunocytokine can constitute about 30% or more by weight of a composition containing one or more single-chain immunocytokines.

Polypeptides may be purified by methods including, but not limited to, affinity chromatography and immunosorbent affinity column. For example, a purified nucleic acid encoding one or more polypeptides that can be used to generate single-chain immunocytokines described herein can constitute about 30% or more by weight of a composition containing one or more amino acid chains that can be used to generate a single-chain immunocytokine described herein. Nucleic acid may be purified by methods including, but not limited to, phenol-chloroform extraction and column purification (e.g., mini-column purification).

Also provided herein are methods and materials for treating a mammal (e.g., a human) in need thereof (e.g., a mammal having a condition that can benefit from activating an immune response within the mammal such as a cancer and/or an infectious disease). In some cases, a composition containing one or more single-chain immunocytokines described herein (e.g., a single-chain immunocytokine that can bind to an IL-2Rβ/γ_{c} polypeptide complex), or nucleic acid encoding one or more polypeptides (e.g., an immunoglobulin heavy chain, an IL-2 polypeptide (or fragment thereof) that can bind an IL-2Rβ/γ_{c} polypeptide complex, and/or an immunoglobulin light chain) that can be used to a generate single-chain immunocytokine described herein, can be used for treating a mammal having a cancer. For example, a composition containing one or more single-chain immunocytokines described herein, or nucleic acid encoding one or more polypeptides that can be used to generate single-chain immunocytokines described herein, can be administered a mammal having a cancer to treat the mammal. In some cases, a composition containing one or more single-chain immunocytokines described herein (e.g., a single-chain immunocytokine that can bind to an IL-2Rβ/γ_{c} polypeptide complex), or nucleic acid encoding one or more polypeptides (e.g., an immunoglobulin heavy chain, an IL-2 polypeptide (or fragment thereof) that can bind an IL-2Rβ/γ_{c} polypeptide complex, and/or an immunoglobulin light chain) that can be used to a generate single-chain immunocytokine described herein, can be used for treating a mammal having an infectious disease. For example, a composition containing one or more single-chain immunocytokines described herein, or nucleic acid encoding one or more polypeptides that can be used to generate single-chain immunocytokines described herein, can be administered a mammal having an infectious disease to treat the mammal.

Any appropriate mammal having, or suspected of having, cancer can be treated as described herein (e.g., by administering a composition a composition containing one or more single-chain immunocytokines that can bind to an IL-2Rβ/γ_{c} polypeptide complex). Examples of mammals that can be treated as described herein include, without limitation, primates (e.g., humans and monkeys), dogs, cats, horses, cows, pigs, sheep, rabbits, mice, and rats. For example, humans having cancer can be treated with a composition containing one or more single-chain immunocytokines that can bind to an IL-2Rβ/γ_{c} polypeptide complex.

When treating a mammal having, or suspected of having, cancer as described herein (e.g., by administering a composition a composition containing one or more single-chain immunocytokines that can bind to an IL-2Rβ/γ_{c} polypeptide complex), the mammal can have, or be suspected of having, any type of cancer. A cancer can include one or more solid tumors. A cancer can be a blood cancer. Examples of cancers that can be treated as described herein include, without limitation, breast cancer, ovarian cancer, prostate cancer, colorectal cancer, brain cancer, skin cancer, kidney cancer, lung cancer (e.g., non-small cell lung cancer), melanoma, oral cancer, bladder cancer, cervical cancer, esophageal cancer, and uterine cancer.

Any appropriate method can be used to identify a mammal (e.g., a human) as having cancer. For example, imaging techniques, biopsy techniques, and/or blood tests can be used to identify mammals (e.g., humans) having cancer.

When treating a mammal having, or suspected of having, an infectious disease as described herein (e.g., by administering a composition a composition containing one or more single-chain immunocytokines that can bind to an IL-2Rβ/γ_{c} polypeptide complex), the mammal can have, or be suspected of having, any type of infectious disease. Examples of infectious diseases that can be treated as described herein include, without limitation, human immunodeficiency virus, malaria, influenza, Ebola, tuberculosis, measles, rabies, Dengue fever, salmonellosis, whooping cough, plague, and West Nile fever.

Any appropriate method can be used to identify a mammal (e.g., a human) as having, or as being at risk of developing, an infectious disease. For example, urine tests, throat swabs, stool samples, and/or blood tests can be used to identify mammals (e.g., humans) having, or at risk of developing, an infectious disease.

Once identified as having a cancer and/or as having, or as being at risk of developing, an infectious disease, a mammal (e.g., a human) can be administered, or instructed to self-administer, a composition containing one or more single-chain immunocytokines described herein (e.g., a single-chain immunocytokine that can bind to an IL-2Rβ/γ_{c} polypeptide complex). In some cases, a composition containing one or more single-chain immunocytokines described herein can be used to reduce the number of cancer cells present in a mammal having cancer. In some cases, a composition containing one or more single-chain immunocytokines described herein can be used to reduce the size (e.g., volume) of a tumor within a mammal having cancer. In some cases, a composition containing one or more single-chain immunocytokines described herein can be used to reduce the number of infectious microbes present in a mammal having an infectious disease.

In some cases, one or more single-chain immunocytokines described herein (e.g., a single-chain immunocytokine that can bind to an IL-2Rβ/γ_{c} polypeptide complex) can be administered to a mammal having a cancer as the sole active ingredients used to treat a mammal having a cancer and/or an infectious disease.

In some cases, where one or more single-chain immunocytokines described herein (e.g., a single-chain immunocytokine that can bind to an IL-2Rβ/γ_{c} polypeptide complex) are administered to a mammal having a cancer, the one or more single-chain immunocytokines described herein can be administered as a combination therapy with one or more additional cancer treatments used to treat a cancer and/or one or more additional treatments used to enhance an immune response. For example, a combination therapy used to treat a cancer can include administering to the mammal (e.g., a human) one or more single-chain immunocytokines described herein and one or more cancer treatments such as surgery, chemotherapy, radiation, administration of a vaccine, adoptive cell transfer, administration of cell therapy, administration of targeted therapy, and/or administration of immunotherapy. For example, a combination therapy used to enhance an immune response can include administering to the mammal (e.g., a human) one or more single-chain immunocytokines described herein and one or more additional treatments used to enhance an immune response such as administration of a vaccine, an adoptive cell transfer, administration of an immune checkpoint inhibitor (e.g., a drug that acts through immune checkpoint blockade), and/or administration of cell therapy.

In some cases, where one or more single-chain immunocytokines described herein (e.g., a single-chain immunocytokine that can bind to an IL-2Rβ/γ_{c} polypeptide complex) are administered to a mammal having an infectious disease, the one or more single-chain immunocytokines described herein can be administered as a combination therapy with one or more additional infectious disease treatments used to treat an infectious disease and/or one or more additional treatments used to enhance an immune response. For example, a combination therapy used to treat an infectious disease can include administering to the mammal (e.g., a human) one or more single-chain immunocytokines described herein and one or more infectious disease treatments such as antibiotics, anti-virals, anti-fungals, and/or anti-parasitics. For example, a combination therapy used to enhance an immune response can include administering to the mammal (e.g., a human) one or more single-chain immunocytokines described herein and one or more additional treatments used to enhance an immune response such as administration of a vaccine, an adoptive cell transfer, administration of an immune checkpoint inhibitor (e.g., a drug that acts through immune checkpoint blockade), and/or administration of cell therapy.

In cases where one or more single-chain immunocytokines described herein are used in combination with one or more additional treatments, the one or more additional treatments can be administered at the same time or independently. For example, one or more single-chain immunocytokines described herein can be administered first, and the one or more additional treatments can be administered second, or vice versa.

In some cases, one or more single-chain immunocytokines described herein (e.g., a single-chain immunocytokine that can bind to an IL-2Rβ/γ_{c} polypeptide complex) can be formulated into a composition (e.g., pharmaceutically acceptable composition) for administration to a mammal in need thereof (e.g., a mammal having a condition that can benefit from activating an immune response within the mammal such as a cancer and/or an infectious disease). For example, a therapeutically effective amount of one or more single-chain immunocytokines described herein can be formulated together with one or more pharmaceutically acceptable carriers (additives) and/or diluents. A pharmaceutical composition can be formulated for administration in any appropriate dosage form. Examples of dosage forms include solid or liquid forms including, without limitation, gums, capsules, tablets (e.g., chewable tablets, and enteric coated tablets), suppository, liquid, enemas, suspensions, solutions (e.g., sterile solutions), sustained-release formulations, delayed-release formulations, pills, powders, and granules. Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol such as Vitamin E TPGS, sodium carboxymethylcellulose, polyacrylates, waxes, polyethylene-polyoxypropylene-block polymers, and wool fat.

A composition (e.g., a pharmaceutical composition) containing one or more single-chain immunocytokines described herein (e.g., a single-chain immunocytokine that can bind to an IL-2Rβ/γ_{c} polypeptide complex) can be designed for oral or parenteral (including subcutaneous, intratumoral, intramuscular, intravenous, and intradermal) administration. When being administered orally, a pharmaceutical composition containing one or more single-chain immunocytokines described herein can be in the form of a pill, tablet, or capsule. Compositions suitable for parenteral administration include aqueous and non-aqueous sterile injection solutions that can contain anti-oxidants, buffers, bacteriostats, and solutes which render the formulation isotonic with the blood of the intended recipient; and aqueous and non-aqueous sterile suspensions which may include suspending agents and thickening agents. The formulations can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

A composition (e.g., a pharmaceutical composition) containing one or more single-chain immunocytokines described herein (e.g., a single-chain immunocytokine that can bind to an IL-2Rβ/γ_{c} polypeptide complex) can be administered locally or systemically. For example, a composition containing one or more single-chain immunocytokines described herein can be administered systemically by an oral administration or by injection to a mammal (e.g., a human).

Effective doses of one or more single-chain immunocytokines described herein (e.g., a single-chain immunocytokine that can bind to an IL-2Rβ/γ_{c} polypeptide complex) can vary depending on the severity of the cancer, the route of administration, the age and general health condition of the subject, excipient usage, the possibility of co-usage with other therapeutic treatments such as use of other agents, and/or the judgment of the treating physician.

An effective amount of a composition containing one or more single-chain immunocytokines described herein (e.g., a single-chain immunocytokine that can bind to an IL-2Rβ/γ_{c} polypeptide complex) can be any amount that can treat a mammal (e.g., a mammal having a cancer and/or having, or at risk of developing, an infectious disease) without producing significant toxicity to the mammal. An effective amount of a single-chain immunocytokine described herein can be any appropriate amount. In some cases, an effective amount of a single-chain immunocytokine described herein can be from about 0.05 milligrams (mg) to about 500 mg per kg of body weight (mg/kg; e.g., from about 0.05 mg/kg to about 400 mg/kg, from about 0.05 mg/kg to about 300 mg/kg, from about 0.05 mg/kg to about 200 mg/kg, from about 0.05 mg/kg to about 100 mg/kg, from about 0.05 mg/kg to about 50 mg/kg, from about 0.5 mg/kg to about 500 mg/kg, from about 1 mg/kg to about 500 mg/kg, from about 50 mg/kg to about 500 mg/kg, from about 100 mg/kg to about 500 mg/kg, from about 200 mg/kg to about 500 mg/kg, from about 300 mg/kg to about 500 mg/kg, from about 400 mg/kg to about 500 mg/kg, from about 0.5 mg/kg to about 400 mg/kg, from about 1 mg/kg to about 300 mg/kg, from about 50 mg/kg to about 200 mg/kg, from about 1 mg/kg to about 100 mg/kg, from about 100 mg/kg to about 200 mg/kg, from about 200 mg/kg to about 300 mg/kg, or from about 300 mg/kg to about 400 mg/kg body weight) of a mammal (e.g., a human). The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and/or severity of the condition (e.g., a cancer) may require an increase or decrease in the actual effective amount administered.

The frequency of administration of a composition containing one or more single-chain immunocytokines described herein (e.g., a single-chain immunocytokine that can bind to an IL-2Rβ/γ_{c} polypeptide complex) can be any frequency that can treat a mammal (e.g., a mammal having a cancer and/or having, or at risk of developing, an infectious disease) without producing significant toxicity to the mammal. For example, the frequency of administration can be from about three times a day to about once a week, from about twice a day to about twice a week, or from about once a day to about twice a week. The frequency of administration can remain constant or can be variable during the duration of treatment. A course of treatment with a composition containing one or more single-chain immunocytokines described herein can include rest periods. For example, a composition containing one or more single-chain immunocytokines described herein can be administered daily over a two-week period followed by a two-week rest period, and such a regimen can be repeated multiple times. As with the effective amount, various factors can influence the actual frequency of administration used for a particular application. For example, the effective amount, duration of treatment, use of multiple treatment agents, route of administration, and/or severity of the condition (e.g., a cancer) may require an increase or decrease in administration frequency.

An effective duration for administering a composition containing one or more single-chain immunocytokines described herein (e.g., a single-chain immunocytokine that can bind to an IL-2Rβ/γ_{c} polypeptide complex) can be any duration that treat a mammal (e.g., a mammal having a cancer and/or having, or at risk of developing, an infectious disease) without producing significant toxicity to the mammal. For example, the effective duration can vary from several days to several weeks, months, or years. In some cases, the effective duration for the treatment of a cancer can range in duration from about one month to about 10 years. Multiple factors can influence the actual effective duration used for a particular treatment. For example, an effective duration can vary with the frequency of administration, effective amount, use of multiple treatment agents, route of administration, and/or severity of the condition being treated.

In some cases, the cancer present within a mammal, and/or the severity of one or more symptoms of the cancer being treated can be monitored. For example, the number of cancer cells and/or the size or a tumor present within a mammal being treated can be monitored. Any appropriate method can be used to determine whether or not the number of cancer cells and/or the size of a tumor present within a mammal is reduced. For example, imaging techniques can be used to assess the number of cancer cells present within a mammal.

Alternatively, the methods and materials described herein can be used for treating a mammal (e.g., a human) having another condition that can benefit from stimulating one or more Effs and/or activating an immune response within the mammal.

The invention will be further described in the following examples, which do not limit the scope of the invention described in the claims.

### EXAMPLES

### Example 1: Engineered Cytokine-Antibody Fusion for Targeted Expansion of Immune Effector T Cells

Antibody-mediated immune bias presents an exciting opportunity for targeted cytokine therapy. Indeed, complexes between IL-2 and the anti-IL-2 antibody S4B6 induce potent anti-tumor activity in mice in the absence of adverse effects typically associated with systemic IL-2 administration. However, clinical translation of a cytokine/antibody complex is hindered by logistical hurdles such as dosing ratio optimization, as well as concerns regarding complex stability, as dissociation would induce dangerous toxicities from the free cytokine. Moreover, S4B6 recognizes mouse IL-2 (mIL-2) and has limited cross-reactivity with human IL-2 (hIL-2).

This Example describes engineering of a clinically relevant single-chain fusion protein that specifically stimulates immune effector T cells to promote anti-cancer immunity.

### Materials and Methods

### Protein Expression and Purification

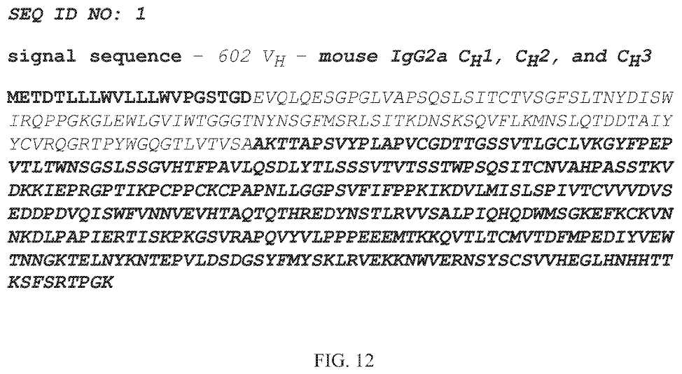

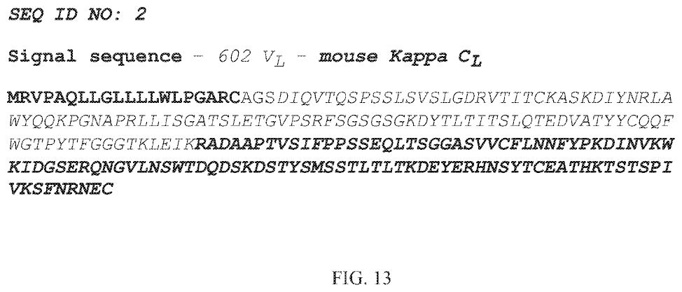

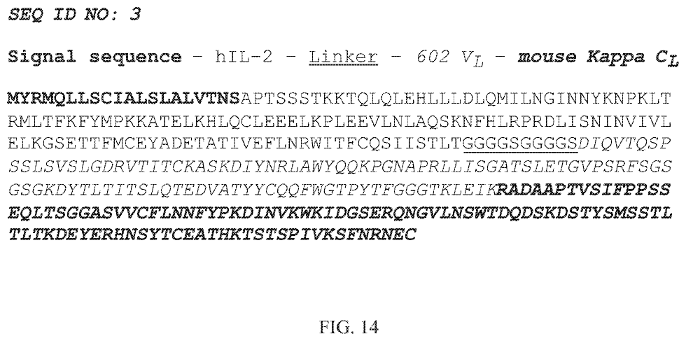

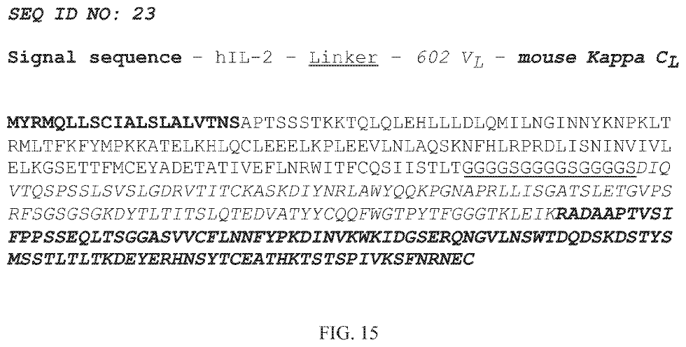

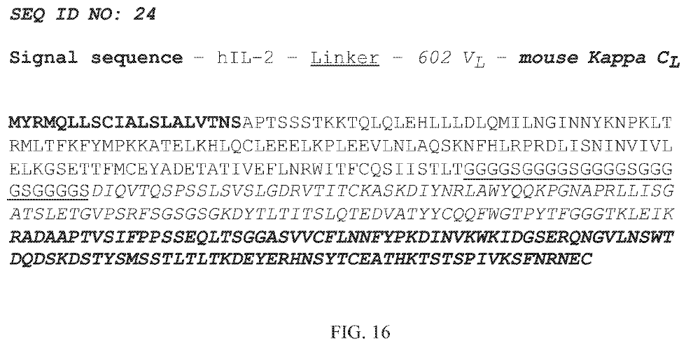

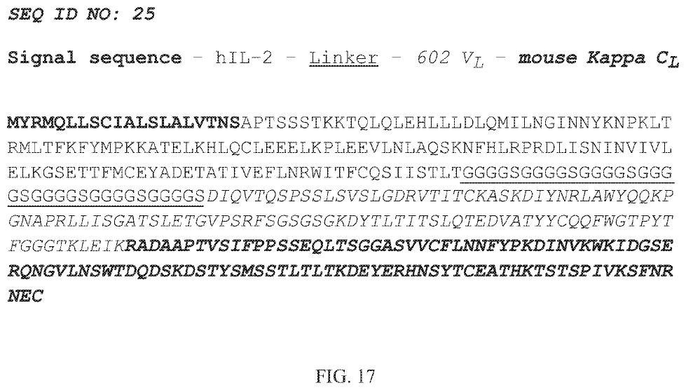

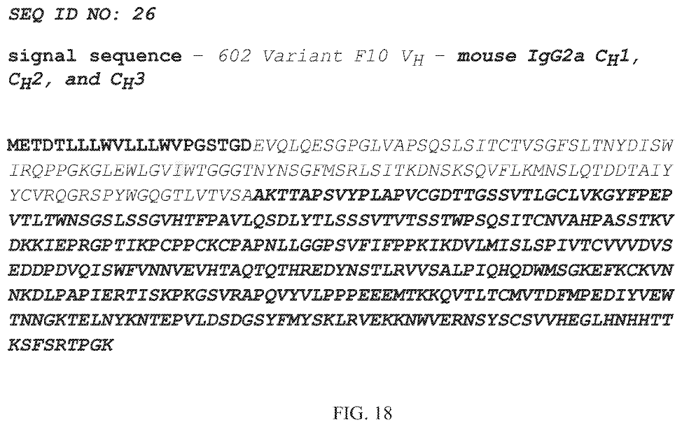

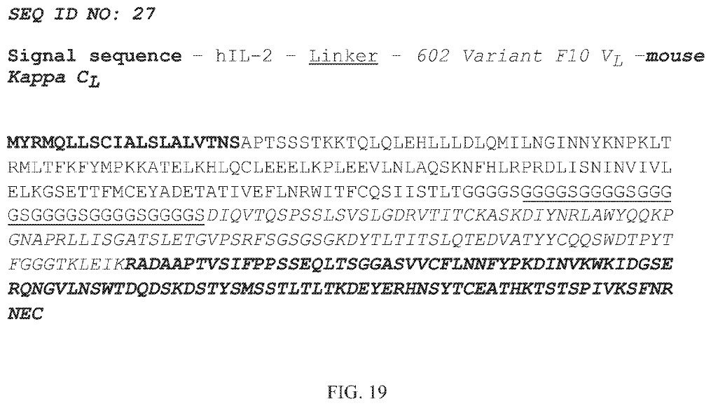

The V_{H} and V_{L} sequences of the 602 antibody were determined by performing PCR amplification on the 602 hybridoma cells. Recombinant antibodies were formulated as mouse immunoglobulin (IgG) 2a kappa isotype to match the parent clone (FIG. 12, SEQ ID NO:1; and FIG. 13, SEQ ID NO:2). The heavy chain (HC) and light chain (LC) of the 602 antibody were separately cloned into the gWiz vector (Genlantis). Antibodies were expressed recombinantly in human embryonic kidney (HEK) 293F cells via transient co-transfection of plasmids encoding the HC and LC. HC and LC plasmids were titrated in small-scale co-transfection tests to determine optimal ratios for large-scale expression. Secreted antibodies were purified from cell supernatants 5 days post-transfection via protein G affinity chromatography followed by size-exclusion chromatography on an FPLC instrument. Purity (>99%) was verified by SDS-PAGE analysis. For wild type 602 immunocytokines (ICs) and variants thereof, the hIL-2 cytokine was fused to the full 602 antibody at the N-terminus of the LC, connected by a flexible (G₄S)₂, (G₄S)₃, (G₄S)₅, or (G₄S)₇ linker to allow for intramolecular interaction (FIG. 14, SEQ ID NO: 3; FIG. 15, SEQ ID NO: 23; FIG. 16, SEQ ID NO: 24; and FIG. 17, SEQ ID NO: 25). These hIL-2-fused 602 LC constructs were also cloned into the gWiz vector (Genlantis). ICs were expressed via transient co-transfection of HEK 293F cells, and purified as described for the 602 antibody. hIL-2 cytokine and hIL-2Rα and hIL-2Rβ receptor extracellular domains containing a C-terminal hexahistidine tag were produced via transient transfection of HEK 293F cells, as described for 602 and the ICs, and purified via Ni-NTA affinity chromatography followed by followed by size-exclusion chromatography on an FPLC instrument. All proteins were stored in HEPES-buffered saline (HBS, 150 mM NaCl in 10 mM HEPES pH 7.3). Purity (>99%) was verified by SDS-PAGE analysis.

For expression of biotinylated hIL-2, hIL-2Rα and hIL-2Rβ, protein containing a C-terminal biotin acceptor peptide (BAP; SEQ ID NO:30) was expressed and purified via Ni-NTA affinity chromatography and then biotinylated with the soluble BirA ligase enzyme in 0.5 mM Bicine pH 8.3, 100 mM ATP, 100 mM magnesium acetate, and 500 mM biotin (Sigma). Excess biotin was removed by size exclusion chromatography on a Superdex 200 column.

### Cell Lines

HEK 293F cells were cultivated in Freestyle 293 Expression Medium (Thermo) supplemented with 10 U/mL penicillin-streptomycin (Gibco). Unmodified YT-1¹⁴ and IL-2Rα⁺ YT-1 human natural killer cells were cultured in RPMI complete medium (RPMI 1640 medium supplemented with 10% fetal bovine serum, 2 mM L-glutamine, minimum non-essential amino acids, sodium pyruvate, 25 mM HEPES, and penicillin-streptomycin [Gibco]) and maintained at 37° C. in a humidified atmosphere with 5% CO₂.

### Bio-Layer Interferometry Binding Studies

For IL-2 versus immunocytokine affinity titration studies, biotinylated human IL-2Rα and IL-2Rβ receptors were immobilized to streptavidin-coated tips for analysis on an Octet® Red96 bio-layer interferometry (BLI) instrument (ForteBio). Less than 5 signal units (nm) of receptor was immobilized to minimize mass transfer effects. Tips were exposed to serial dilutions of hIL-2, 602 Ab, hIL-2 in complex with 602 Ab (formed by pre-incubating hIL-2 and 602 Ab in a 2:1 ratio for 30 minutes at room temperature), 602 ICs, or a control IC (comprised of hIL-2 fused to the N terminus of the light chain of an irrelevant anti-fluorescein antibody [4-4-20], with a framework sequence identical to that of 602), in a 96-well plate for 300 seconds and dissociation was measured for 600 seconds. An irrelevant protein (the human monoclonal antibody trastuzumab) was included in a reference well to subtract non-specific binding. Surface regeneration for all interactions was conducted using 15 seconds exposure to 0.1 M glycine pH 3.0. Experiments were carried out in PBSA (phosphate-buffered saline, pH 7.3 plus 0.1% bovine serum albumin [BSA, Thermo Fisher Scientific]) at 25° C. Data was visualized and processed using the Octet® Data Analysis software version 7.1 (Molecular Devices). Equilibrium titration curve fitting and K_{D} value determination were implemented using GraphPad Prism, assuming all binding interactions to be first order. Experiments were reproduced twice with similar results.

### YT-1 Cell STAT5 Phosphorylation Studies

Approximately 2×10⁵ IL-2Rα⁻ YT-1 or IL-2Rα⁺ YT-1 cells were plated in each well of a 96-well plate and resuspended in RPMI complete medium containing serial dilutions of hIL-2, hIL-2/602 complex, or the various ICs. Complexes were formed by incubating 602 Ab with IL-2 at a 1:1 molar ratio of antibody or antibody fragment to hIL-2 for 30 minutes at room temperature. Cells were stimulated for 15 minutes at 37° C. and immediately fixed by addition of formaldehyde to 1.5% and 10 minute incubation at room temperature. Permeabilization of cells was achieved by resuspension in ice-cold 100% methanol for 30 minutes at 4° C. Fixed and permeabilized cells were washed twice with FACS buffer (phosphate-buffered saline [PBS] pH 7.2 containing 0.1% BSA [Thermo Fisher Scientific]) and incubated with Alexa Fluor® 647-complexed anti-STAT5 pY694 (BD Biosciences) diluted in FACS buffer for 2 hours at room temperature. Cells were then washed twice in FACS buffer and MFI was determined on a CytoFLEX flow cytometer (Beckman-Coulter). Dose-response curves were fitted to a logistic model and half-maximal effective concentrations (EC₅₀s) were calculated using GraphPad Prism data analysis software after subtraction of the mean fluorescence intensity (MFI) of unstimulated cells and normalization to the maximum signal intensity. Experiments were conducted in triplicate and performed three times with similar results.

### Immune Cell Subset Expansion Studies in Mice

To determine whether immune complexes bias the immune response to favor effector cell expansion, 12 week old C57BL/6 mice (3 per cohort) were injected i.p. with PBS or hIL-2/602 or mIL-2/S4B6 complexes (prepared by pre-incubating hIL-2 [eBioscience] with 602 or S4B6 at a 2:1 cytokine:antibody molar ratio in PBS for 30 minutes) daily for 4 days. Mice were sacrificed on day 5 by cervical dislocation and spleens were harvested. Single-cell suspensions were prepared by mechanical homogenization and absolute count of splenocytes was assessed for each spleen by automated cell counter (Vicell, Beckman Coulter). Cells were resuspended in PBS and subsequently stained for 30 minutes on ice with fluorophore-complexed anti-mouse monoclonal antibodies (mAbs) for phenotyping of T_{Reg} (CD4⁺IL-2Rα⁺Foxp3⁺) or CD8⁺ effector T cells (CD8⁺) using BV605-complexed anti-CD4 (Biolegend, clone RM4-5), PeCy7-complexed anti-IL-2Rα (eBioscience, clone PC61.5), PerCP-Cy5.5-complexed anti-CD8 (eBioscience, clone 53-6.72), and mAbs. Fixable Blue Dead Cell Stain Kit (Life Techonologies) was used to assess live cells. Cells were then washed twice with FACS buffer (1% BSA, 1% Sodium Azide) and fixed in FoxP3 Transcription Factor Fixation/Permeabilization Buffer (eBioscience) for 30 minutes on ice. After two washes in Permeabilization Buffer (eBioscience), T_{Reg} cells were stained with FITC-complexed anti-mouse/rat Foxp3 mAb (eBioscience, clone FJK-16s) for 1 hour on ice. Two final washes were conducted in Permeabilization Buffer and cells were resuspended in FACS buffer for flow cytometric analysis on an LSRII (BD Biosciences). Data were analyzed using FlowJo X software (Tree Star). Abundance of MP CD8⁺ effector T cells relative to PBS control cells is presented. Experiments were performed three times with similar results.

### Tumor Therapy Studies in Mice.

For mouse syngeneic melanoma models, 1×10⁶B16F10 tumor cells were inoculated s.c. into C57BL/6 male mice (6-8 wks old, n=8 per treatment cohort). Beginning on day 4 post-inoculation, mice were injected i.p. twice weekly with PBS or hIL-2/602 complex (prepared by pre-incubating hIL-2 with 602 at a 2:1 cytokine:antibody molar ratio for 30 minutes). Tumor size and body weight were tracked daily to evaluate efficacy of the cytokine/antibody complex. Mice were sacrificed on Day 20 for tumor excision and analysis.

Generation of a Mutagenic Yeast-Displayed Library of 602 scFv Variants (EP602)

The single chain variable fragment (scFv) version of the 602 antibody (consisting of the heavy chain variable domain followed by the light chain variable domain, separated by a (G₄S)₃ linker) was displayed on yeast through fusion of the N-terminus of the variable domain of the heavy chain to the C-terminus of Aga2, with the two separated by a (G₄S)₃ linker, along with 3C and factor Xa cleavage sites. A C-terminal c-Myc tag was included for detection.

A targeted error prone library that mutagenized the CDR1 and CDR3 of both the heavy and light chain variable domains was generated to preserve existing IL-2 interactions, while allowing for potentially beneficial, conservative alterations in binding. The four targeted CDRs were amplified by error prone PCR using Taq polymerase (New England Biolabs), with standard 1× Taq buffer, 2 mM manganese(II) chloride, 7 mM magnesium chloride, 0.2 mM of dATP and dGTP, 1 mM dCTP and dTTP, 0.5 μM of each primer, and 0.2 ng/μL of template. Following five amplification cycles, the mix was transferred and diluted 1:5 into fresh mix lacking template. Another five cycles proceeded, and the transfer and dilution was repeated twice more, with the final transfer undergoing 20 total amplification cycles.

The five framework sequences adjacent to the targeted CDRs were amplified using Phusion High-Fidelity DNA polymerase (Thermo Scientific). These framework fragments were assembled with the neighboring mutagenized fragments by sequential, pairwise overlap extension PCR with Phusion polymerase. The final, assembled fragments contained the full 602 scFv as well as homologous sequences (≥97 nt) on both ends that would provide overlap with the cut yeast display vector, pCT3CBN.

The cut vector and mutagenized fragments were assembled by yeast homologous recombination, following electroporation of EBY100 yeast in the presence of the linear backbone and fragment, as previously described.^{1,2} The library yielded 1.4×10⁷ transformants, and was grown in SDCAA media for 48 hours prior to passaging, followed by induction in SGCAA 24 hours later at an initial OD of 1. A sample of the recombined plasmids was extracted by yeast plasmid miniprep (Zymogen) to verify proper insertion of the fragment into the backbone. A productive error rate—i.e. DNA mutations that resulted in changes to the amino acid sequence—of approximately 6% was observed.

### EP602 Library Selections

Each round of selection stained and sorted enough yeast to ensure 10-fold coverage of the remaining clones. Yeast selected from each round were grown overnight at 30° C. in SDCAA liquid media (pH 4.5) for 2 days, followed by induction in SGCAA liquid media (pH 4.5) for 2 days at 20° C.

The naïve EP602 library was debulked in the first round of magnetic-activated cell selection (MACS) by eliminating variants specific to Alexa Fluor 647-conjugated streptavidin (SA-AF647) (Thermo Scientific), and selecting for variants that bound IL-2. All staining was done in PBE solution (phosphate-buffered saline pH 7.2, 0.1% BSA, and 1 mM ethylenediaminetetraacetic acid (EDTA)). In the first MACS step, the yeast were incubated with 20 μg/mL SA-AF647 for 1 hr at 4° C., washed, and then incubated with 1:20 anti-Cy5/anti-Alexa Fluor 647 microbeads (Miltenyi Biotec) for 20 minutes at 4° C., washed, and then run over a LS MACS separation column (Miltenyi Biotec), according to the manufacturer's protocol. The yeast that flowed through the column (i.e. non-SA-AF647 binders) were collected, and prepared for the second MACS step. Biotinylated IL-2 was mixed with SA-647 (4:1 molar ratio; Thermo Fisher Scientific) diluted in PBE and incubated for 15 minutes to form tetramer, and the yeast were incubated with the tetramer for 2 hours at 4° C., and then washed and incubated with anti-Cy5/anti-Alexa Fluor 647 microbeads as before. Again, the yeast were run over a LS MACS separation column, but in this step, cells eluted after removing the column from the magnet were collected, and were grown as described before being induced for the next round of selection.

The second round of selection isolate full-length 602 scFv variants by using MACS to select for the presence of c-Myc. Yeast were incubated with a 1:100 dilution of Alexa Fluor 647-conjugated anti-c-Myc antibody (clone 9B11, Cell Signaling Technologies) in PBE for 2 hours at 4° C., incubated with anti-Cy5/anti-Alexa Fluor 647 microbeads, and run over an LS MACS separation column. Eluted cells were collected as described above.

The final three rounds of selection were performed by fluorescence-activated cell sorting (FACS) on a FACSymphony S6 cell sorter (Becton Dickinson), using decreasing amounts of IL-2 in the presence of a large excess of IL-2Rα to select for the variants that competitively block IL-2 interaction with the IL-2Rα subunit. The first of these competition FACS selections used 50 nM IL-2 with 1.5 μM IL-2Rα, and the second and third both used 30 nM IL-2 and 0.3 μM IL-2Rα, but unlike the first two selections, the third selection selected for variants with low off-rates by incubating with IL-2, washing, and then incubating with IL-2Rα at room temp for 2 hr (allowing for IL-2 dissociation). In all FACS selections, only the variants in the top 5% for IL-2 binding were collected.

Yeast Surface scFv Binding Studies.

Yeast displaying scFvs (2×10⁵ per well) were plated in a 96-well plate and incubated in PBE buffer containing biotinylated IL-2 in the presence or absence of IL-2Rα for 4 hr at room temperature. Cells were then washed and stained with a 1:200 dilution of AlexaFluor 647-complexed streptavidin (Thermo Fisher Scientific) in PBSA for 15 minutes at 4° C. After a final wash, cells were analyzed for target binding using a CytoFLEX flow cytometer (Beckman Coulter). Background-subtracted and normalized binding curves were fitted to a first-order binding model and equilibrium dissociation constant (K_{D}) values were determined using GraphPad Prism software.

### Results

602 Immunocytokine is Robustly Produced from Mammalian Cells.

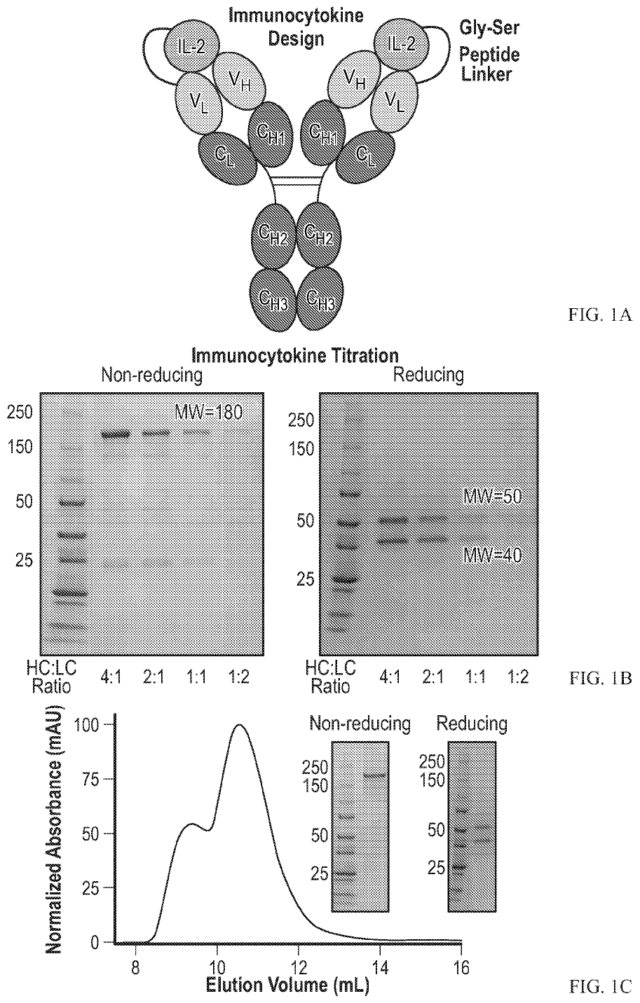

To combine the potency of cytokines with the pharmacokinetically favorable properties of antibodies, hIL-2 was fused to the cytokine-biasing 602 antibody (FIG. 1A) to create an immunocytokine (IC). A rapid small-scale HEK 293F cell transfection assay was developed to optimize immunocytokine expression. Cells were transfected in 6-well plates with pre-defined ratios of HC and IL-2-fused LC plasmid DNA. After 3-day incubation, secreted protein was captured from the supernatant with protein G resin, eluted with 0.1 M glycine pH 2.0, and analyzed via SDS-PAGE. This assay was validated using an immunocytokine comprised of hIL-2 fused to a mouse IgG2a antibody at the LC N-terminus. Titration of the HC:LC ratio revealed the optimal expression conditions (FIG. 1B). Immunocytokine expression was scaled up in HEK 293F cells and the secreted protein was purified via protein G chromatography followed by size-exclusion chromatography. 602 IC was purified to homogeneity on an FPLC instrument (FIG. 1C).

### Immunocytokine Selectively Biases Receptor Binding and Cell Signaling.

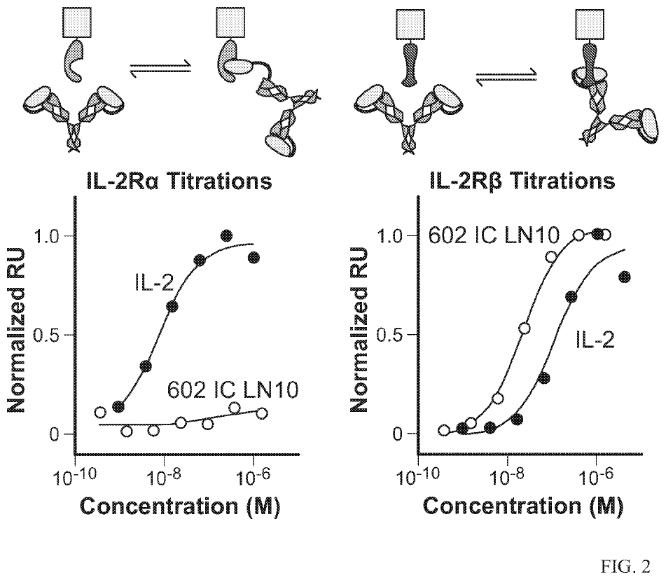

Using bio-layer interferometry on the Octet® platform, the human IL-2Rα and IL-2Rβ receptors were immobilized and measured binding of soluble ICs was compared to the hIL-2 cytokine. The 602 IC did not interact with IL-2Rα due to antibody blockade but IL-2Rβ binding was enhanced relative to untethered hIL-2 (FIG. 2), confirming functional formation of the intramolecular antibody/cytokine complex.

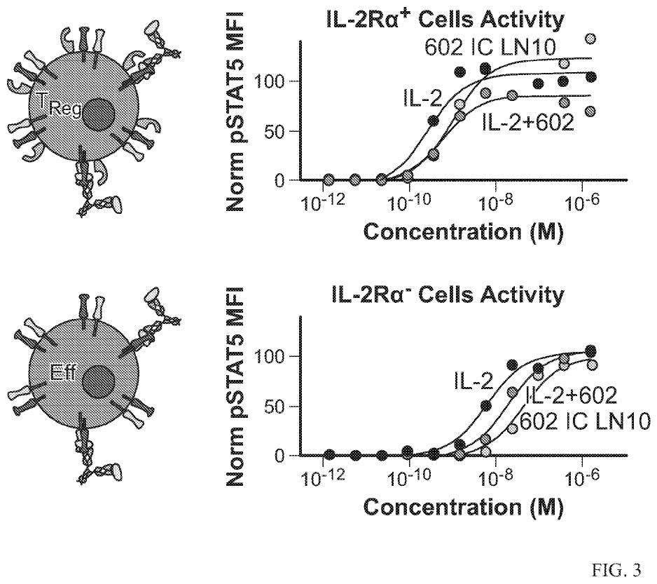

The downstream signaling response to IC stimulation on IL-2-responsive immune cells was analyzed to assess IC function. To characterize the immune bias mediated by the engineered IC, the human YT-1 natural killer (NK) cell line, which inducibly expresses IL-2Rα, was used. Flow cytometry-based studies were performed to quantify STAT5 phosphorylation elicited by IL-2, cytokine/antibody complexes (prepared by pre-incubating IL-2 and 602 antibody in a 1:1 stoichiometrically equal molar ratio), or 602 IC on uninduced IL-2Rα″ versus induced IL-2Rα⁺ YT-1 cells as a surrogate for Eff versus T_{Reg} activation. Untethered IL-2 stimulates both IL-2Rα⁺ and IL-2Rα⁻ cells, and the hIL-2/602 complex exhibits impaired activity on both IL-2Rα⁺ and IL-2Rα″ YT-1 cells. It was observed that the 602 IC effectively recapitulated the cell signaling properties of the hIL-2/602 complex (FIG. 3), demonstrating that, like the mixed complex, 602 IC biases cytokine signaling toward Effs.

### IL-2/602 Complexes Expand Effs and Inhibit Tumor Growth In Vivo.

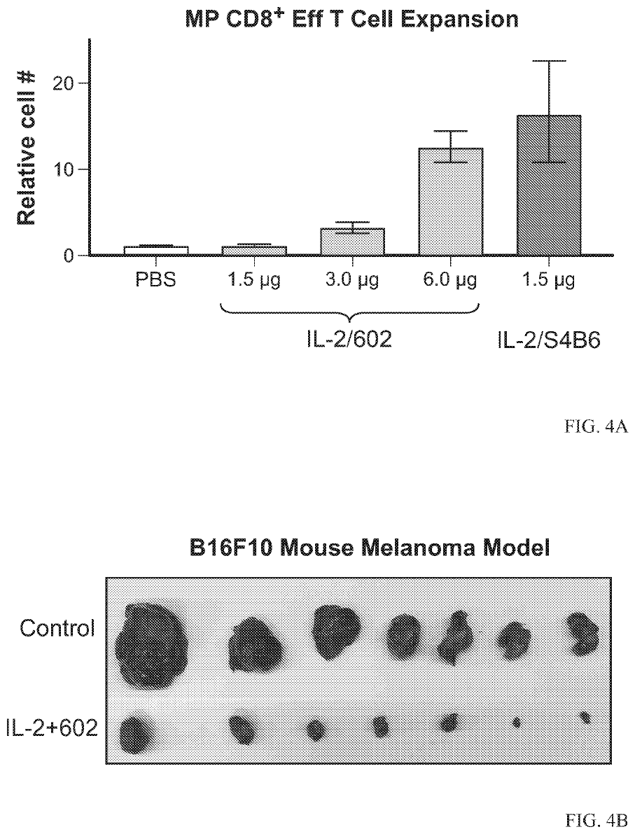

To determine whether IL-2/602 complexes expand Effs (specifically MP CD8⁺ effector T cells) in live animals, mice were injected with either PBS or various concentrations of hIL-2/602 or mIL-2/S4B6 complexes. It was observed that hIL-2/602 complexes expand MP CD8⁺ effector T cells, albeit less potently than mIL-2/S4B6 complexes (FIG. 4A). It was further shown that twice weekly administration of hIL-2/602 complexes significantly inhibit tumor growth (FIG. 4B) in a mouse syngeneic melanoma model (B16F10).

### Modifying Linker Length Optimizes 602 Immunocytokine Production and Function.

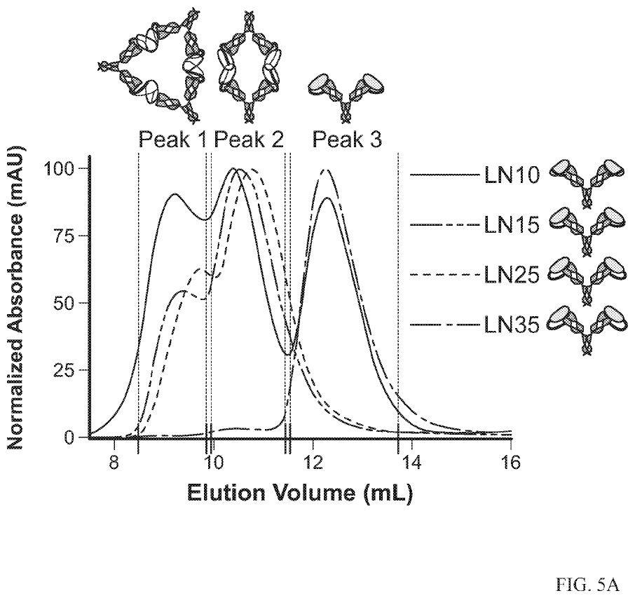

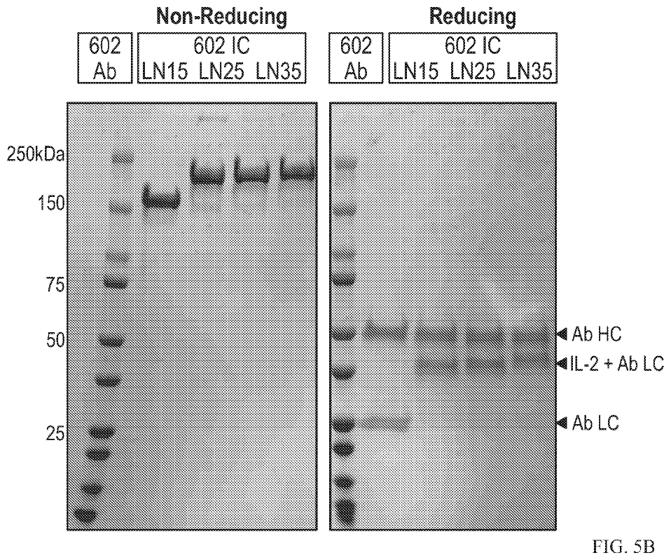

To improve the expression, stability, and function of the 602 IC, the length of the intramolecular linker between the IL-2 cytokine and the 602 antibody within the immunocytokine was adjusted. Specifically, 602 IC variants with 15 (SEQ ID NO:23, 602 IC LN15), 25 (SEQ ID NO:24, 602 IC LN25), and 35 (SEQ ID NO:25, 602 IC LN35) amino acid linkers were created. All constructs were expressed via transient co-transfection of HEK 293F cells with the 602 heavy chain and the IL-2-fused 602 light chain with the appropriate length linker. As shown in FIG. 5A, 3 peaks were evident by size exclusion chromatography. The first 2 peaks (Peaks 1 and 2) correspond to high molecular weight oligomers, whereas Peak 3 corresponds to the monomeric IC. As linker length increased, the fraction of monomer also increased, and 602 IC LN35 was nearly exclusively in the monomeric state. We thus proceeded to further characterize immunocytokines based on the 35 amino acid linker construction. All 602 IC variants migrated at the expected molecular weights via non-reducing and reducing SDS-PAGE analysis (FIG. 5B).

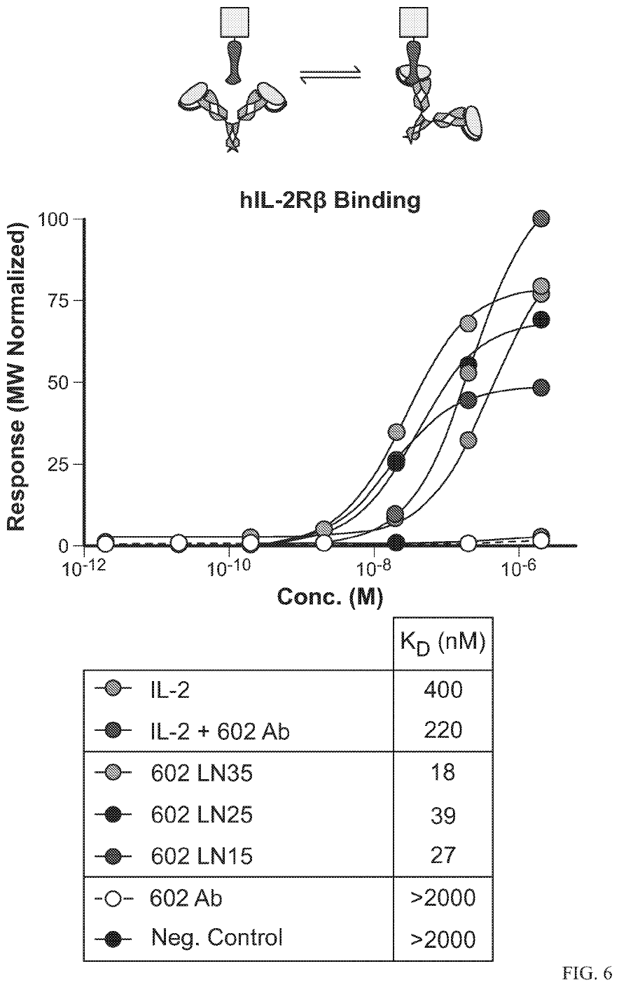

Proper assembly was verified and the binding properties of 602 IC variants were interrogated via bio-layer interferometry studies using the Octet® platform. Human IL-2Rβ was immobilized, and binding of IL-2, 602 antibody, the IL-2+602 Ab complex (prepared by pre-incubating IL-2 and 602 antibody in a 2:1 stoichiometric ratio for 30 minutes at room temperature), 602 IC variants, and an unrelated negative control protein was measured. As expected based on the intramolecular interactions between IL-2 and 602 within the immunocytokine, the IC interactions with IL-2Rβ were enhanced compared to unconjugated IL-2, demonstrating proper assembly and biophysical behavior of 602 ICs (FIG. 6).

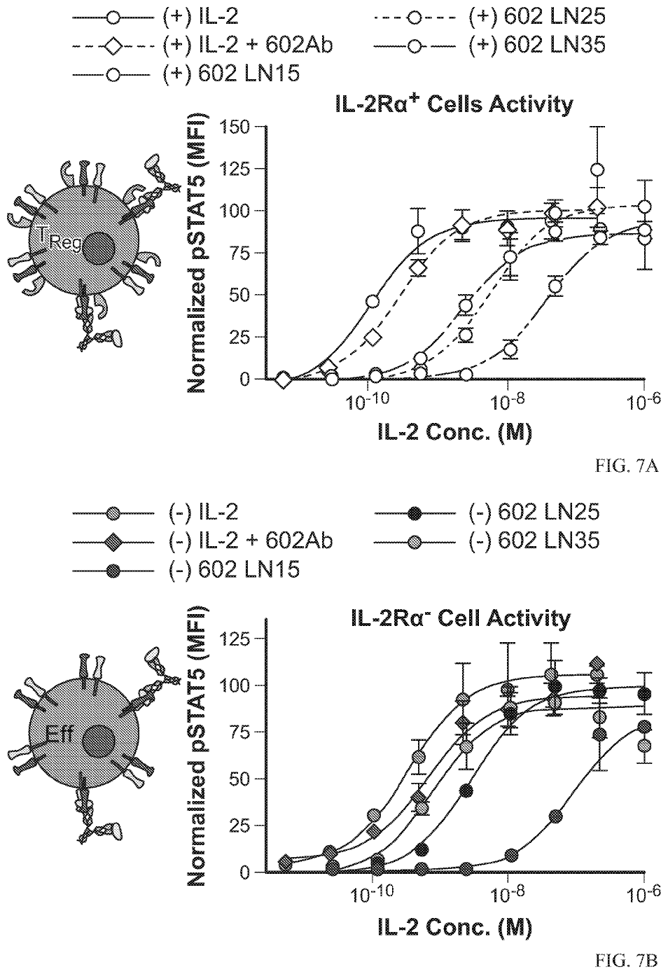

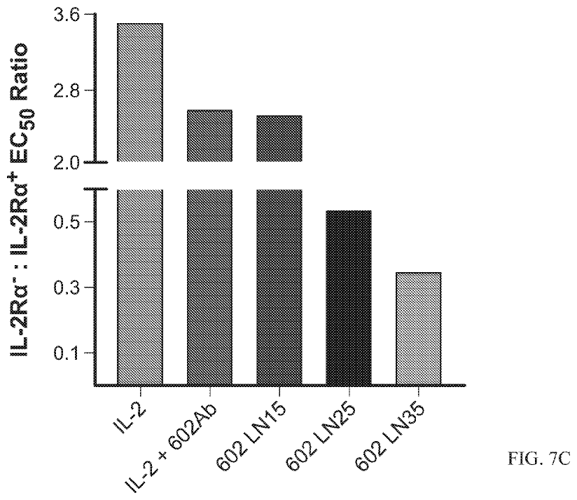

The downstream signaling response to stimulation with the IC variants was evaluated on IL-2-responsive immune cells to assess function. Human YT-1 NK cells with and without IL-2Rα expression were used to mimic responses on T_{Reg} versus immune effector cells. IL-2-mediated STAT5 phosphorylation was quantified by flow cytometry following stimulation with either IL-2, IL-2+602 Ab complex prepared by pre-incubating IL-2 and 602 antibody in a 1:1 stoichiometric ratio for 30 minutes at room temperature), or 602 IC variants (FIG. 7). Untethered IL-2 showed a 3-4-fold bias toward activation of IL-2Rα⁺ T_{Reg}-like cells, whereas the lead IC, 602 IC LN35, reversed this bias to favor IL-2Rα″ Eff-like cells. Note that the bias mediated by IC exceeded that mediated by the IL-2+602 Ab complex.

### Engineered Version of 602 IC has Improved Bias Toward Effector Cells.

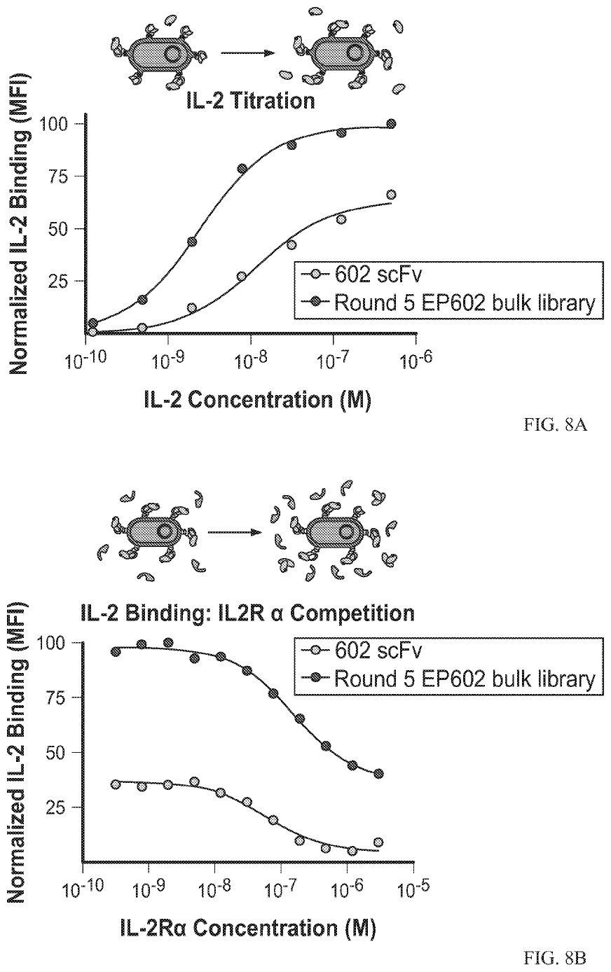

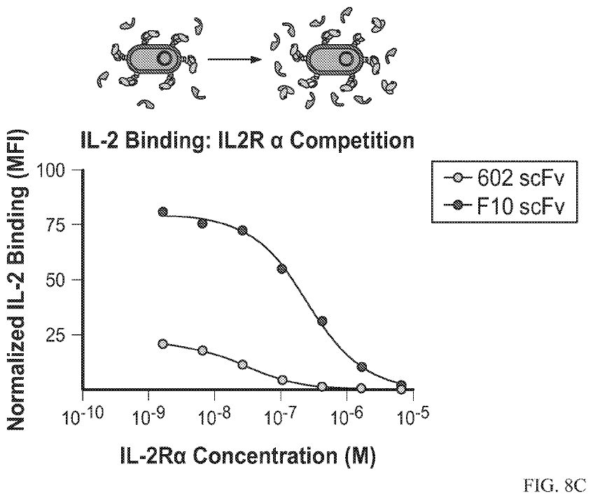

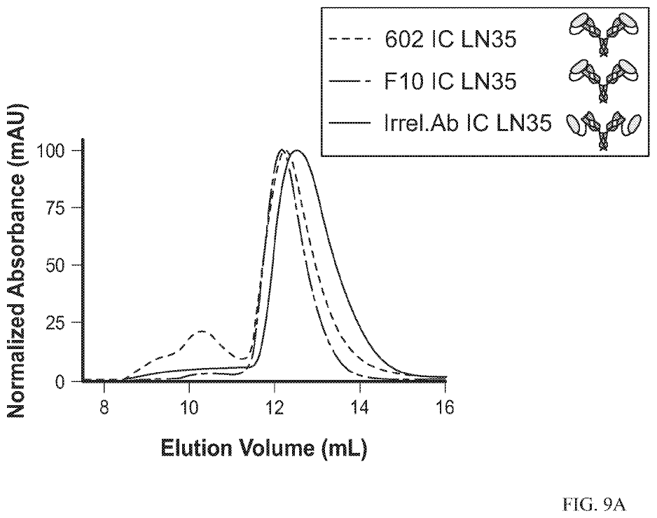

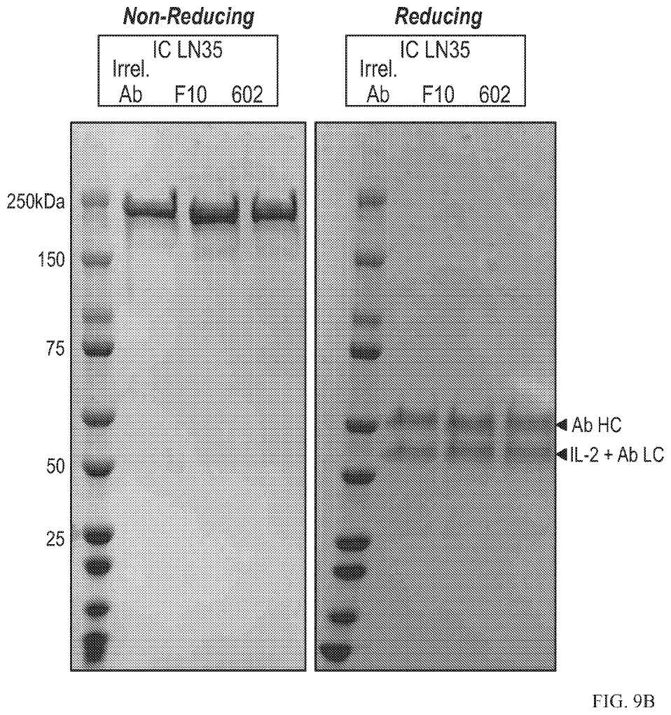

To further skew the activities of the 602 IC toward Effs over T_{Reg} cells, an error-prone mutagenic library was generated that randomized the complementarity-determining loops (CDRs) of the 602 variable heavy and light chains. This library was then evolved against human IL-2 through iterative rounds of magnetic-activated cell sorting (MACS) and fluorescence-activated cell sorting (FACS), and clones that successfully outcompeted soluble IL-2Rα for cytokine engagement were selected. After 5 rounds of selection, the evolved library showed significantly improved binding to IL-2 (FIG. 8A) and enhanced competition with the IL-2Rα receptor subunit (FIG. 8B). Individual clones from this library were characterized, and it was found that the most effective mutant to be a variant denoted F10 (SEQ ID NO:28 and SEQ ID NO:29). As shown in FIG. 8C, the F10 antibody (in single-chain variable fragment [scFv] format) demonstrated stronger binding to IL-2 and more potent competition with IL-2Rα compared to the parent 602 scFv. The F10 antibody was reformatted as an IC, using the optimized 35 amino acid linker. F10 IC LN35 was expressed from HEK 293F cells via transient co-transfection of the antibody heavy chain and the IL-2-fused antibody light chain. As shown in FIG. 9A, the majority of secreted protein was monomeric. An IC construct consisting of an irrelevant antibody fused to IL-2 with a 35 amino acid linker was also prepared as an experimental control (Irrel. Ab IC LN35). F10 IC LN35 and the irrelevant antibody migrated at the expected molecular weights via non-reducing and reducing SDS-PAGE (FIG. 9B).

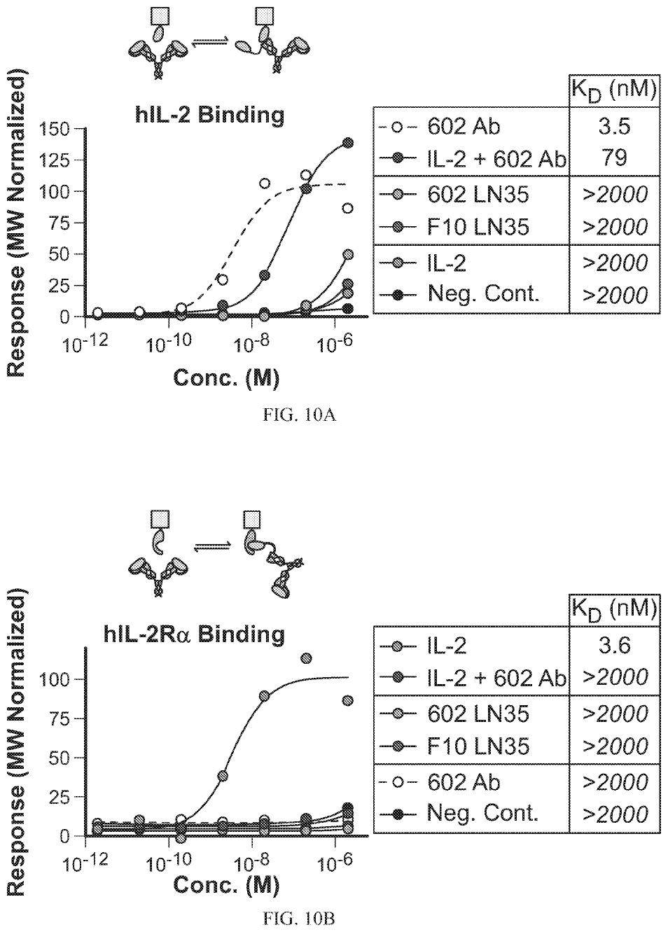

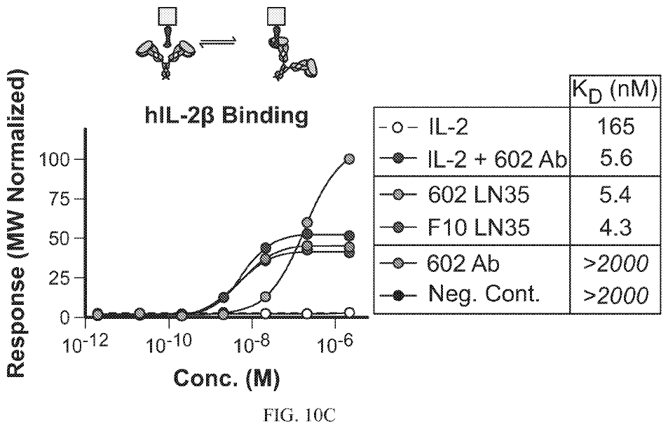

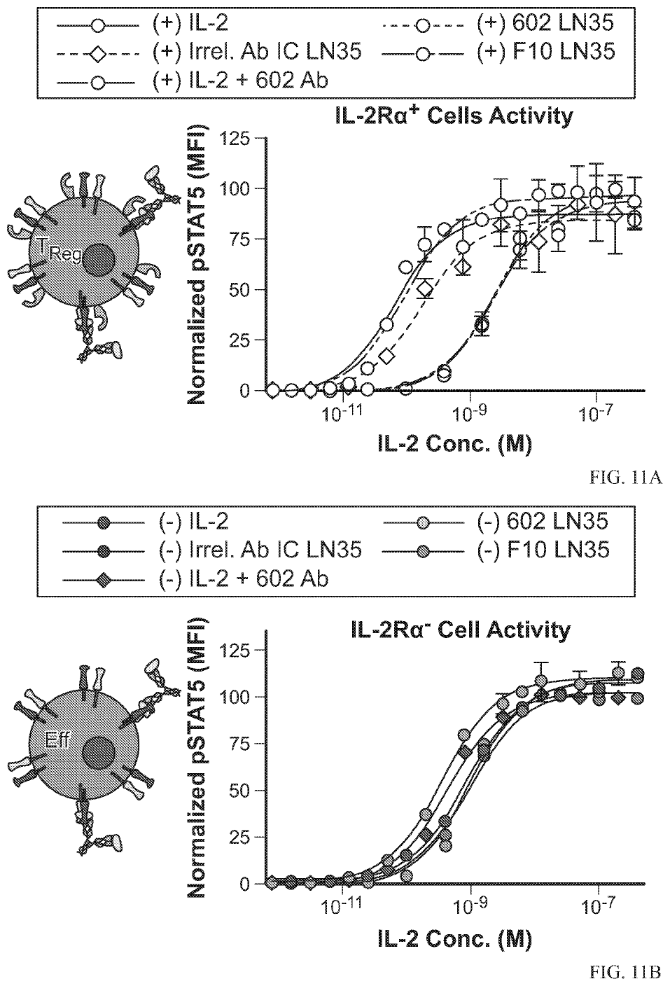

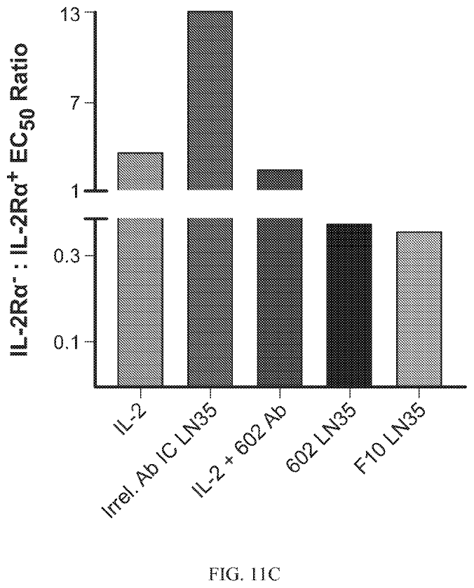

To verify the assembly and proper binding behavior of F10 IC LN35, bio-layer interferometry experiments were performed using an Octet® instrument. Human IL-2, IL-2Rα, and IL2Rβ receptors were immobilized, and binding of 602 antibody, the IL-2+602 Ab complex (prepared by pre-incubating IL-2 and 602 antibody in a 2:1 stoichiometric ratio for 30 minutes at room temperature), 602 IC LN35, F10 IC LN35, and an unrelated negative control protein was measured. IC interaction with IL-2 was greatly attenuated compared to the unconjugated 602 antibody due to competition from intramolecular interactions within the IC (FIG. 10A). Moreover, like 602 IC LN35, F10 IC LN35 showed minimal binding to IL-2Rα due to antibody blockade of IL-2 cytokine binding, and F10 IC LN35 was more competitive with the receptor than 602 IC LN35, as designed. (FIG. 10B). Binding to IL-2Rβ was enhanced compared to unconjugated IL-2 for both 602 IC LN35 and F10 IC LN35 (FIG. 10C). Collectively, these binding studies demonstrate the intramolecular assembly and biophysical functionality of the engineered IC variant F10 IC LN35.

The superior bias of F10 LN35 IC toward IL-2Rα″ Eff-like cells compared to IL-2Rα⁺ T_{Reg}-like cells was demonstrated using the YT-1 human NK cell line. YT-1 with and without IL-2Rα were stimulated with either IL-2, irrelevant antibody Ab IC LN35, IL-2+602 Ab complex (prepared by pre-incubating IL-2 and 602 antibody in a 1:1 stoichiometric ratio for 30 minutes at room temperature), 602 IC LN35, or F10 LN35. STAT5 phosphorylation was detected via flow cytometry as a readout for IL-2-induced signaling (FIG. 11). Whereas IL-2 and the control irrelevant antibody Ab IC LN35 showed significant bias toward IL-2Rα⁺ T_{Reg}-like cells over IL-2Rα″ Eff-like cells, F10 IC LN35 reversed this preference, and slightly outperformed the parent 602 IC LN35. Both F10 IC LN35 and 602 IC LN35 exhibited significantly stronger preference for Eff-like versus T_{Reg}-like cells compared to the IL-2+602 complex.

### Summary

These results demonstrate that IL2/602 immunocytokines can stimulate immune effector cell activity, and can be used to treat a mammal having cancer and/or an infectious disease.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### Sequence Listing Free Text:

signal sequence-602 V_{H}-mouse IgG2a C_{H}1, C_{H}2, and C_{H}3
SEQ ID NO: 1
METDTLLLWVLLLWVPGSTGDEVQLQESGPGLVAPSQSLSITCTVSGFSLTNYDISW
IRQPPGKGLEWLGVIWTGGGTNYNSGFMSRLSITKDNSKSQVFLKMNSLQTDDTAIY
YCVRQGRTPYWGQGTLVTVSAAKTTAPSVYPLAPVCGDTTGSSVTLGCLVKGYFPEPVT
LTWNSGSLSSGVHTFPAVLQSDLYTLSSSVTVTSSTWPSQSITCNVAHPASSTKVDKKI
EPRGPTIKPCPPCKCPAPNLLGGPSVFIFPPKIKDVLMISLSPIVTGVVVDVSEDDPDV
QISWFVNNVEVHTAQTQTHREDYNSTLRITSALPIQHQDWMSGKEFKCKVNNKDLPAPI
ERTISKPKGSVRAPQVYVLPPPEEEMTKKQVTLTCMVTDFMPEDIYVEWTNNGKTELNY
KNTEPVLDSDGSYFMYSKLRVEKKNWVERNSYSGSWHEGLHNHHTTKSFSRTPGK
Signal sequence-602 V_{L}-mouse Kappa C_{L}
SEQ ID NO: 2
MRVPAQLLGLLLLWLPGARCAGSDIQVTQSPSSLSVSLGDRVTITCKASKDIYNRL
AWYQQKPGNAPRLLISGATSLETGVPSRFSGSGSGKDYTLTITSLQTEDVATYYCQQF
WGYPYYYGGGYKLEIKRADAAPTFSIFPPSSEQLTSGGASWCFLNNFYPKDINVKWKID
GSERQNGVLNSWTDQDSKDSTYSMSSTLTLTKDEYERHNSYTCEATHKTSTSPIVKSFN
RNEC
Signal sequence-hIL-2-Linker-602 V_{L}-mouse Kappa C_{L}
SEQ ID NO: 3
MYRMQLLSCIALSLALVTNS

[symbol]

[symbol]

[symbol]

[symbol]

[symbol]

[symbol]

[symbol]

GGGGSGGGGSDIQVTQSPSS
LSVSLGDRVTITCKASKDIYNRLAWYQQKPGNAPRLLISGATSLETGVPSRFSGSGSG
KDYTLTITSLQTEDVATYYCQQFWGTPYTFGGGTKLEIKRADAAPTVSIFPPSSEQLTS
GGASWCFLNNFYPKDINVKWKIDGSERQNGVLNSWTDQDSKDSTYSMSSTLTLTKDEYE
RHNSYTCEATHKTSTSPIVKSFNRNEC
602 HC variable domain
SEQ ID NO: 4
EVQLQESGPGLVAPSQSLSITCTVSGFSLTNYDISWIRQPPGKGLEWLGVIWTGGGTN
YNSGFMSRLSITKDNSKSQVFLKMNSLQTDDTAIYYCVRQGRTPYWGQGTLVTVSA
602 HC constant domain
SEQ ID NO: 5
AKTTAPSVYPLAPVCGDTTGSSVTLGCLVKGYFPEPVTLTWNSGSLSSGVHTFPAVLQ
SDLYTLSSSVTVTSSTWPSQSITCNVAHPASSTKVDKKIEPRGPTIKPCPPCKCPAPN
LLGGPSVFIFPPKIKDVLMISLSPIVTCVVVDVSEDDPDVQISWFVNNVEVHTAQTQT
HREDYNSTLRVVSALPIQHQDWMSGKEFKCKVNNKDLPAPIERTISKPKGSVRAPQV
YVLPPPEEEMTKKQVTLTCMVTDFMPEDIYVEWTNNGKTELNYKNTEPVLDSDGSY
FMYSKLRVEKKNWVERNSYSCSVVHEGLHNHHTTKSFSRTPGK
signal sequence
SEQ ID NO: 6
METDTLLLWVLLLWVPGSTGD
signal sequence
SEQ ID NO: 7
MRVPAQLLGLLLLWLPGARC
signal sequence
SEQ ID NO: 8
MYRMQLLSCIALSLALVTNS
human IL-2
SEQ ID NO: 9
APTSSSTKKTQLQLEHLLLDLQMILNGINNYKNPKLTRMLTFKFYMPKKATELKHLQ
CLEEELKPLEEVLNLAQSKNFHLRPRDLISNINVIVLELKGSETTFMCEYADETATI
VEFLNRWITFCQSIISTLT
602 LC variable domain
SEQ ID NO: 10
DIQVTQSPSSLSVSLGDRVTITCKASKDIYNRLAWYQQKPGNAPRLLISGATSLETGV
PSRFSGSGSGKDYTLTITSLQTEDVATYYCQQFWGTPYTFGGGTKLEIK
602 LC constant domain
SEQ ID NO: 11
RADAAPTVSIFPPSSEQLTSGGASVVCFLNNFYPKDINVKWKIDGSERQNGVLNSWT
DQDSKDSTYSMSSTLTLTKDEYERHNSYTCEATHKTSTSPIVKSFNRNEC
linker
SEQ ID NO: 12
GGGGSGGGGS
linker
SEQ ID NO: 13
GGGGSGGGGSGGGGS
linker
SEQ ID NO: 14
GGGGSGGGGSGGGGSGGGGS
linker
SEQ ID NO: 15
GGGGSGGGGSGGGGSGGGGSGGGGS
linker
SEQ ID NO: 16
GGGGSGGGGSGGGGSGGGGSGGGGSGGGGS
linker
SEQ ID NO: 17
GGGGSGGGGSGGGGSGGGGSGGGGSGGGGSGGGGS
linker
SEQ ID NO: 18
GGGGSGGGGSGGGGSGGGGSGGGGSGGGGSGGGGSGGGGS
linker
SEQ ID NO: 19
GGGGSGGGGSGGGGSGGGGSGGGGSGGGGSGGGGSGGGGSGGGGS
linker
SEQ ID NO: 20
GGGGSGGGGSGGGGSGGGGSGGGGSGGGGSGGGGSGGGGSGGGGSGGGGS
linker
SEQ ID NO: 21
GGGGSGGGGSGGGGSGGGGSGGGGSGGGGSGGGGSGGGGSGGGGSGGGGSGGGGS
linker
SEQ ID NO: 22
GGGGSGGGGSGGGGSGGGGSGGGGSGGGGSGGGGSGGGGSGGGGSGGGGSGGGG
SGGGGS
Signal sequence-hIL-2-Linker-602 V_{L}-mouse Kappa C_{L}
SEQ ID NO: 23
MYRMQLLSCIALSLALVTNS

[symbol]

[symbol]

[symbol]

[symbol]

[symbol]

[symbol]

[symbol]

GGGGSGGGGSGGGGSDIQV
TQSPSSLSVSLGDRVTITCKASKDIYNRLAWYQQKPGNAPRLLISGATSLETGVPSRFS
GSGSGKDYTLTITSLQTEDVATYYCQQFWGTPYTFGGGTKLEIKRADAAPTVSIFPPSS
EQLTSGGASWCFLNNFYPKDINVKWKIDGSERQNGVLNSWTDQDSKDSTYSMSSTLTLT
KDEYERHNSYTCEATHKTSTSPIVKSFNRNEC
Signal sequence-hIL-2-Linker-602 V_{L}-mouse Kappa C_{L}
SEQ ID NO: 24
MYRMQLLSCIALSLALVTNS

[symbol]

[symbol]

[symbol]

[symbol]

[symbol]

[symbol]

[symbol]

GGGGSGGGGSGGGGSGGG
GSGGGGSDIQVTQSPSSLSVSLGDRVTITCKASKDIYNRLAWYQQKPGNAPRLLISGA
TSLETGVPSRFSGSGSGKDYTLTITSLQTEDVATYYCQQFWGTPYTFGGGTKLEIKRA
DAAPTVSIFPPSSEQLTSGGASWCFLNNFYPKDINVKWKIDGSERQNGVLNSWTDQDSK
DSTYSMSSTLTLTKDEYERHNSYTCEATHKTSTSPIVKSFNRNEC
Signal sequence-hIL-2-Linker-602 V_{L}-mouse Kappa C_{L}
SEQ ID NO: 25
MYRMQLLSCIALSLALVTNS

[symbol]

[symbol]

[symbol]

[symbol]

[symbol]

[symbol]

[symbol]

GGGGSGGGGSGGGGSGGG
GSGGGGSGGGGSGGGGSDIQVTQSPSSLSVSLGDRVTITCKASKDIYNRLAWYQQKP
GNAPRLLISGATSLETGVPSRFSGSGSGKDYTLTITSLQTEDVATYYCQQFWGTPYTF
GGGYYYLEYKRADAAPTVSIFPPSSEQLTSGGASWCFLNNFYPKDINVKWKIDGSERQ
NGVLNSWTDQDSKDSTYSMSSTLTLTKDEYERHNSYTCEATHKTSTSPIVKSFNRNEC
signal sequence-602 Variant F10 V_{H}-mouse IgG2a C_{H}1, C_{H}2, and C_{H}3
SEQ ID NO: 26
METDTLLLWVLLLWVPGSTGDEVQLQESGPGLVAPSQSLSITCTVSGFSLTNYDISW
IRQPPGKGLEWLGVIWTGGGTNYNSGFMSRLSITKDNSKSQVFLKMNSLQTDDTAIY
YCVRQGRSPYWGQGYLNYNSAAKTTAPSVYPLAPVCGDTTGSSVTLGCLVKGYFPEP
VTLTWNSGSLSSGVHTFPAVLQSDLYTLSSSVTVTSSTWPSQSITCNVAHPASSTKV
DKKIEPRGPTIKPCPPCKCPAPNLLGGPSVFIFPPKIKDVEMISLSPIVTCVWDVSE
DDPDVQISWFVNNVEVHTAQTQTHREDYNSTLRWSALPIQHQDWMSGKEFKCKVNNK
DLPAPIERTISKPKGSVRAPQVYVLPPPEEEMTKKQVTLTCMVTDFMPEDIYVEWTN
NGKTELNYKNTEPVLDSDGSYFMYSKLRVEKKNWVERNSYSCSWHEGLHNHHTTKSF
SRTPGK
Signal sequence-hIL-2-Linker-602 Variant F10 V_{L}-mouse Kappa C_{L}
SEQ ID NO: 27
MYRMQLLSCIALSLALVTNS

[symbol]

[symbol]

[symbol]

[symbol]

[symbol]

[symbol]

[symbol]

GGGGSGGGGSGGGGSGGG
GSGGGGSGGGGSGGGGSDIQVTQSPSSLSVSLGDRVTITCKASKDIYNRLAWYQQKP
GNAPRLLISGATSLETGVPSRFSGSGSGKDYTLTITSLQTEDVATYYCQQSWDTPYTF
GGGYKEEIKRADAAPTVSIFPPSSEQLTSGGASWCFLNNFYPKDINVKWKIDGSERQN
GVLNSWTDQDSKDSTYSMSSTLTLTKDEYERHNSYTCEATHKTSTSPIVKSFNRNEC
602 Variant F10 V_{H}
SEQ ID NO: 28
EVQLQESGPGLVAPSQSLSITCTVSGFSLTNYDISWIRQPPGKGLEWLGVIWTGGGTN
YNSGFMSRLSITKDNSKSQVFLKMNSLQTDDTAIYYCVRQGRSPYWGQGTLVTVSA
602 Variant F10 V_{L}
SEQ ID NO: 29
DIQVTQSPSSLSVSLGDRVTITCKASKDIYNRLAWYQQKPGNAPRLLISGATSLETG
VPSRFSGSGSGKDYTLTITSLQTEDVATYYCQQSWDTPYTFGGGTKLEIK
Biotin acceptor peptide
SEQ ID NO: 30
LNDIFEAQKIEWHE

1. A single-chain immunocytokine comprising:
an immunoglobulin heavy chain comprising a variable domain having at least 80% identity to an amino acid sequence set forth in SEQ ID NO:4 or having at least 80% identity to an amino acid sequence set forth in SEQ ID NO:28;
an IL-2 polypeptide, wherein said IL-2 polypeptide can bind a polypeptide complex comprising an interleukin-2 receptor-β (IL-2Rβ) polypeptide and a common gamma chain (γc) polypeptide (an IL-2Rβ/γ_{c} polypeptide complex); and
an immunoglobulin light chain comprising a variable domain having at least 80% identity to an amino acid sequence set forth in SEQ ID NO:10 or having at least 80% identity to an amino acid sequence set forth in SEQ ID NO:29;
wherein said single-chain immunocytokine binds to said IL-2Rβ/γ_{c} polypeptide complex.

2. (canceled)

3. (canceled)

4. The single-chain immunocytokine of claim 1, wherein said immunoglobulin heavy chain comprises a γ heavy chain constant domain.

5. (canceled)

6. (canceled)

7. The single-chain immunocytokine of claim 1, wherein said immunoglobulin heavy chain comprises a signal sequence.

8. The single-chain immunocytokine of claim 7, wherein said signal sequence comprises an amino acid sequence set forth in SEQ ID NO:6.

9. The single-chain immunocytokine of claim 1, wherein said immunoglobulin heavy chain comprises an amino acid sequence set forth in SEQ ID NO:1 or SEQ ID NO:26.

10-13. (canceled)

14. The single-chain immunocytokine of claim 1, wherein said immunoglobulin light chain comprises a κ light chain constant domain.

15. (canceled)

16. (canceled)

17. The single-chain immunocytokine of claim 12, wherein said immunoglobulin light chain comprises a signal sequence.

18. The single-chain immunocytokine of claim 17, wherein said signal sequence comprises an amino acid sequence set forth in SEQ ID NO:7 or SEQ ID NO:8.

19. The single-chain immunocytokine of claim 1, wherein said immunoglobulin light chain comprises an amino acid sequence set forth in SEQ ID NO:2.

20. The single-chain immunocytokine of claim 1, wherein said IL-2 polypeptide and said immunoglobulin light chain are a fusion polypeptide.

21-27. (canceled)

28. The single-chain immunocytokine of claim 20, wherein said IL-2 polypeptide and said immunoglobulin light chain are fused via a linker.

29. The single-chain immunocytokine of claim 28, wherein said linker is a peptide linker comprising from 10 to 60 amino acids.

30. The single-chain immunocytokine of claim 29, wherein said linker is a (Gly₄Ser)₂ linker.

31. (canceled)

32. (canceled)

33. The single-chain immunocytokine of claim 20, wherein said immunoglobulin light chain comprises an amino acid sequence set forth in any one of SEQ ID NO:3, SEQ ID NO:23, SEQ ID NO:24, SEQ ID NO:25, or SEQ ID NO:27.

34. The single-chain immunocytokine of claim 1, wherein said single-chain immunocytokine has a half-life of from about 5 minutes to about 6 months.

35. The single-chain immunocytokine of claim 1, wherein said single-chain immunocytokine has an affinity for said IL-2Rβ polypeptide of from about 300 nM K_{D} to about 1 pM K_{D}.

36-38. (canceled)

39. A nucleic acid encoding the single-chain immunocytokine of claim 1.

40. The nucleic acid of claim 39, said nucleic acid comprising a first nucleic acid and a second nucleic acid, wherein said first nucleic acid can encode said an immunoglobulin heavy chain, and wherein said second nucleic acid can encode said IL-2 polypeptide fused to said immunoglobulin light chain.

41. A method for treating a mammal having cancer, said method comprising:
administering a composition comprising the single-chain immunocytokine of claim 1 to said mammal.

42. (canceled)

43. The method of claim 41, where said cancer is selected from the group consisting of breast cancer, ovarian cancer, prostate cancer, brain cancer, skin cancer, kidney cancer, lung cancer, melanoma, oral cancer, bladder cancer, colorectal cancer, cervical cancer, esophageal cancer, and uterine cancer.

44. (canceled)

45. A method for stimulating effector cells in a mammal, said method comprising:
administering a composition comprising the single-chain immunocytokine of claim 1 to said mammal.

46. (canceled)

47. A method for treating a mammal having an infectious disease, said method comprising:
administering a composition comprising the single-chain immunocytokine of claim 1 to said mammal.

48. (canceled)

49. The method of claim 47, wherein said infectious disease is selected from the group consisting of human immunodeficiency virus, malaria, influenza, Ebola, tuberculosis, measles, rabies, Dengue fever, salmonellosis, whooping cough, plague, and West Nile fever.

50. (canceled)


SEQUENCE LISTING (30 sequences)

SEQ ID NO: 1 - protein, 465 aa, Artificial
  Recombinant antibody heavy chain polypeptide sequence
METDTLLLWVLLLWVPGSTGDEVQLQESGPGLVAPSQSLSITCTVSGFSLTNYDISWIRQPPGKGLEWLGVIWTGGGTNYNSGFMSRLSITKDNSKSQVFLKMNSLQTDDTAIYYCVRQGRTPYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 2 - protein, 237 aa, Artificial
  Recombinant antibody light chain polypeptide sequence
MRVPAQLLGLLLLWLPGARCAGSDIQVTQSPSSLSVSLGDRVTITCKASKDIYNRLAWYQQKPGNAPRLLISGATSLETGVPSRFSGSGSGKDYTLTITSLQTEDVATYYCQQFWGTPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - protein, 377 aa, Artificial
  Immunocytokine light chain polypeptide sequence
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSDIQVTQSPSSLSVSLGDRVTITCKASKDIYNRLAWYQQKPGNAPRLLISGATSLETGVPSRFSGSGSGKDYTLTITSLQTEDVATYYCQQFWGTPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 4 - protein, 114 aa, Artificial
  Immunoglobulin 602 heavy chain variable domain polypeptide sequence
EVQLQESGPGLVAPSQSLSITCTVSGFSLTNYDISWIRQPPGKGLEWLGVIWTGGGTNYNSGFMSRLSITKDNSKSQVFLKMNSLQTDDTAIYYCVRQGRTPYWGQGTLVTVSA

SEQ ID NO: 5 - protein, 330 aa, Artificial
  Immunoglobulin 602 heavy chain constant domain polypeptide sequence
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 6 - protein, 21 aa, Artificial
  Signal sequence
METDTLLLWVLLLWVPGSTGD

SEQ ID NO: 7 - protein, 20 aa, Artificial
  Signal sequence
MRVPAQLLGLLLLWLPGARC

SEQ ID NO: 8 - protein, 20 aa, Artificial
  Signal sequence
MYRMQLLSCIALSLALVTNS

SEQ ID NO: 9 - protein, 133 aa, Homo sapiens [sequence omitted]

SEQ ID NO: 10 - protein, 107 aa, Artificial
  Immunoglobulin 602 light chain variable domain polypeptide sequence
DIQVTQSPSSLSVSLGDRVTITCKASKDIYNRLAWYQQKPGNAPRLLISGATSLETGVPSRFSGSGSGKDYTLTITSLQTEDVATYYCQQFWGTPYTFGGGTKLEIK

SEQ ID NO: 11 - protein, 107 aa, Artificial
  Immunoglobulin 602 light chain constant domain polypeptide sequence
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 12 - protein, 10 aa, Artificial
  Polypeptide linker
GGGGSGGGGS

SEQ ID NO: 13 - protein, 15 aa, Artificial
  Polypeptide linker
GGGGSGGGGSGGGGS

SEQ ID NO: 14 - protein, 20 aa, Artificial
  Polypeptide linker
GGGGSGGGGSGGGGSGGGGS

SEQ ID NO: 15 - protein, 25 aa, Artificial
  Polypeptide linker
GGGGSGGGGSGGGGSGGGGSGGGGS

SEQ ID NO: 16 - protein, 30 aa, Artificial
  Polypeptide linker
GGGGSGGGGSGGGGSGGGGSGGGGSGGGGS

SEQ ID NO: 17 - protein, 35 aa, Artificial
  Polypeptide linker
GGGGSGGGGSGGGGSGGGGSGGGGSGGGGSGGGGS

SEQ ID NO: 18 - protein, 40 aa, Artificial
  Polypeptide linker
GGGGSGGGGSGGGGSGGGGSGGGGSGGGGSGGGGSGGGGS

SEQ ID NO: 19 - protein, 45 aa, Artificial
  Polypeptide linker
GGGGSGGGGSGGGGSGGGGSGGGGSGGGGSGGGGSGGGGSGGGGS

SEQ ID NO: 20 - protein, 50 aa, Artificial
  Polypeptide linker
GGGGSGGGGSGGGGSGGGGSGGGGSGGGGSGGGGSGGGGSGGGGSGGGGS

SEQ ID NO: 21 - protein, 55 aa, Artificial
  Polypeptide linker
GGGGSGGGGSGGGGSGGGGSGGGGSGGGGSGGGGSGGGGSGGGGSGGGGSGGGGS

SEQ ID NO: 22 - protein, 60 aa, Artificial
  Polypeptide linker
GGGGSGGGGSGGGGSGGGGSGGGGSGGGGSGGGGSGGGGSGGGGSGGGGSGGGGSGGGGS

SEQ ID NO: 23 - protein, 382 aa, Artificial
  Immunocytokine light chain polypeptide sequence
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSDIQVTQSPSSLSVSLGDRVTITCKASKDIYNRLAWYQQKPGNAPRLLISGATSLETGVPSRFSGSGSGKDYTLTITSLQTEDVATYYCQQFWGTPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 24 - protein, 392 aa, Artificial
  Immunocytokine light chain polypeptide sequence
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSDIQVTQSPSSLSVSLGDRVTITCKASKDIYNRLAWYQQKPGNAPRLLISGATSLETGVPSRFSGSGSGKDYTLTITSLQTEDVATYYCQQFWGTPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 25 - protein, 402 aa, Artificial
  Immunocytokine light chain polypeptide sequence
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSGGGGSDIQVTQSPSSLSVSLGDRVTITCKASKDIYNRLAWYQQKPGNAPRLLISGATSLETGVPSRFSGSGSGKDYTLTITSLQTEDVATYYCQQFWGTPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 26 - protein, 465 aa, Artificial
  Immunocytokine heavy chain polypeptide sequence
METDTLLLWVLLLWVPGSTGDEVQLQESGPGLVAPSQSLSITCTVSGFSLTNYDISWIRQPPGKGLEWLGVIWTGGGTNYNSGFMSRLSITKDNSKSQVFLKMNSLQTDDTAIYYCVRQGRSPYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 27 - protein, 402 aa, Artificial
  Immunocytokine light chain polypeptide sequence
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSGGGGSDIQVTQSPSSLSVSLGDRVTITCKASKDIYNRLAWYQQKPGNAPRLLISGATSLETGVPSRFSGSGSGKDYTLTITSLQTEDVATYYCQQSWDTPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 28 - protein, 114 aa, Artificial
  Immunoglobulin 602 F10 variant heavy chain variable domain polypeptide sequence
EVQLQESGPGLVAPSQSLSITCTVSGFSLTNYDISWIRQPPGKGLEWLGVIWTGGGTNYNSGFMSRLSITKDNSKSQVFLKMNSLQTDDTAIYYCVRQGRSPYWGQGTLVTVSA

SEQ ID NO: 29 - protein, 107 aa, Artificial
  Immunoglobulin 602 F10 variant light chain variable domain polypeptide sequence
DIQVTQSPSSLSVSLGDRVTITCKASKDIYNRLAWYQQKPGNAPRLLISGATSLETGVPSRFSGSGSGKDYTLTITSLQTEDVATYYCQQSWDTPYTFGGGTKLEIK

SEQ ID NO: 30 - protein, 14 aa, Artificial
  biotin acceptor peptide
LNDIFEAQKIEWHE